(19) United States
(12) Patent Application Publication
(10) Pub. No.: US 2026/0077179 A1
(43) Pub. Date: Mar. 19, 2026

(54) IMPLANTABLE MEDICAL DEVICE HAVING A LINKER AND METHODS THEREOF

(71) Applicant: Puzzle Medical Devices Inc., Montreal (CA)

(72) Inventors: Gabriel GEORGES, Sherbrooke (CA); François TRUDEAU, Quebec (CA); Yves-Antoine CRÊTE, Montreal (CA); Philippe CORMIER, Montreal (CA); Marc BLONDIN, Montreal (CA); Philippe GÉNÉREUX, Basking Ridge, NJ (US); Maxime ROCHON, Québec (CA)

(21) Appl. No.: 19/255,847

(22) Filed: Jun. 30, 2025

Related U.S. Application Data
(60) Provisional application No. 63/695,844, filed on Sep. 17, 2024.
(60) Provisional application No. 63/766,136, filed on Mar. 3, 2025.

(51) Int. Cl. A61M 60/861; A61M 60/139; A61M 60/165; A61M 60/473; A61M 60/495; A61M 60/50; A61M 60/865

(52) U.S. Cl. CPC A61M 60/861; A61M 60/139; A61M 60/165; A61M 60/473; A61M 60/495; A61M 60/50; A61M 60/865; A61M 2205/04

(57) ABSTRACT

Embodiments described herein include a modular transcatheterly implantable medical device. The medical device includes a plurality of assemblable blood pumps. Each one of the blood pumps includes a pump unit and an electrical wire configured for the pump unit to be electrically operated through the electrical wire. The medical device also includes an anchor having a first end portion and a second end portion, and the anchor is configured to intraluminally assemble to and intraluminally unassemble from the blood pumps. The medical device also includes a catheter. The catheter is configured to be in a slidable relationship with each one of the electrical wires for intraluminally assembling the blood pumps to the anchor and for intraluminally unassembling the blood pumps from the anchor. The medical device further includes a flexible linker configured to couple the catheter and the anchor.

### CROSS-REFERENCE TO RELATED APPLICATIONS

The present patent application claims the benefit of and priority to U.S. Provisional Application No. 63/695,844, filed Sep. 17, 2024, and U.S. Provisional Application No. 63/766,136, filed Mar. 3, 2025, the contents of each of which are incorporated herein by reference in their entirety.

### FIELD

This disclosure generally relates to an implantable medical device having a linker and methods thereof.

### BACKGROUND

Implantable medical devices have revolutionized modern medicine by providing effective solutions for treating a wide range of medical conditions. These devices are designed to be placed within the body notably to improve or restore physiological function(s), replace damaged anatomical structures, and/or deliver therapeutic interventions. Among these, transcatheterly implantable medical devices stand out as minimally invasive alternatives to traditional surgical implants, offering shorter recovery times and reduced procedural risks. Such devices may be designed as two or more components thereof that are assemblable together and/or unassemblable from each other in vivo.

However, the successful use of transcatheterly implantable medical devices in narrow and/or tortuous anatomical structures, such as those with acute angles or multiple curves, present significant challenges. The vascular system is a typical example of a narrow and/or tortuous lumen with complex pathways. Anatomical variations, including vessel narrowing, calcification, and acute angulations can impede or prevent the use of transcatheterly implantable medical devices and increase the risk of procedural complications.

These challenges are compounded when, for example, the transcatheterly implantable medical device is required to be precisely positioned and/or anchored, as slight deviations can potentially lead to device malfunction or failure. For example, using a transcatheterly implantable medical device in an angled blood vessel, such as adjacent or in the aortic arch, generally requires a balance of flexibility and rigidity of the device to be used appropriately in vivo. Mispositioning and/or misaligning the transcatheterly implantable medical device may result in the implantable medical device abutting the lumen wall, hindering or preventing its appropriate operation or functioning. This may also be the case for non-transcatheterly implantable medical devices.

There is therefore one or more needs in the field of implantable medical devices.

### BRIEF DESCRIPTION OF THE DRAWINGS

In order that this disclosure be readily understood, at least some selected embodiments thereof are illustrated by way of examples in the accompanying drawings. Accordingly, the accompanying drawings are illustrative in nature only and are not intended to be construed and interpreted as limiting the extent of the subject matter protected by the claims herein.

It is noted that like reference characters identify similar or equivalent feature(s) throughout the drawings. If present in the claims, reference character(s) is/are provided only to make claims easier to comprehend and are not intended to be construed and interpreted as limiting the extent of the subject matter protected by the claims herein. Further, the feature(s) illustrated throughout the drawings is/are not necessarily drawn to scale.

To help understand the drawings and figures, element(s) and/or feature(s) that may be optional to the technology disclosed may be represented in dashed lines.

FIG. 1 is a schematic representation of an implantable medical device provided with a linker, and optionally including an assemblable medical device, in accordance with an aspect of this disclosure. The implantable medical device may optionally include multiples assemblable medical devices.

FIGS. 2-3 are schematic representations of an implantable medical device, such as one of FIG. 1, at least partially implanted in an aorta of a subject's body, in accordance with one or more embodiments.

FIGS. 4-5 are schematic representations of an implantable medical device, such as one of FIG. 1, at least partially implanted in through the left ventricle of a subject's body, in accordance with one or more embodiments.

FIG. 6 is a perspective view of an implantable medical device, such as one of FIG. 1, in accordance with one or more embodiments.

FIG. 7 is a perspective view of a second medical device component of the implantable medical device of FIG. 6 that is assembled to three optional assemblable medical devices, in accordance with one or more embodiments.

FIG. 8 is a perspective view of an end portion of a first medical device component of the implantable medical device of FIG. 6 having an end portion of an optional assemblable medical device superposed to it for the sake of clarity, in accordance with one or more embodiments. The double-headed arrow indicates a possible movement of the end portion of the optional assemblable medical device.

FIG. 9 is a perspective view of the second medical device component of the implantable medical device of FIG. 7 with the three optional assemblable medical devices having various configurations, in accordance with one or more embodiments.

FIG. 10 is a perspective view of the first medical device component of the implantable medical device of FIG. 8 having an end portion that is optionally atraumatic and optionally removable, in accordance with one or more embodiments.

FIG. 11 is a perspective view of the first medical device component of the implantable medical device of FIG. 10 having the end portion optionally removed, in accordance with one or more embodiments.

FIG. 12 is a perspective view of the second medical device component of the implantable medical device of FIG. 7 without any assemblable medical device, in accordance with one or more embodiments.

FIG. 13 is a top view of the second medical device component of the implantable medical device of FIG. 7 without any assemblable medical device, in accordance with one or more embodiments.

FIGS. 14-15 are perspective views of another second medical device component of an implantable medical device, such as one of FIG. 1, without or with three assemblable medical devices assembled to the second medical device component, respectively, in accordance with one or more embodiments.

FIGS. 16-17 are perspective views of another second medical device component of an implantable medical device, such as one of FIG. 1, without or with three assemblable medical devices assembled to the second medical device component, respectively, in accordance with one or more embodiments.

FIG. 18 is a side view of another second medical device component of an implantable medical device, such as one of FIG. 1, without any assemblable medical device, in accordance with one or more embodiments. The remaining components of the implantable medical device are omitted in FIG. 18.

FIGS. 19-22 are perspective views of another second medical device component of an implantable medical device, such as one of FIG. 1, that is provided with three optional assemblable medical devices, in accordance with one or more embodiments.

FIGS. 23-24 are photographs of an implantable medical device, such as one of FIG. 1, without and with optional assemblable medical devices, respectively, that is at least partially implanted in a descending aorta and a left subclavian artery of a mock anatomical vascular system, in accordance with an aspect of this disclosure and one or more embodiments.

FIGS. 25-26 are schematic representations of methods of implanting and explanting, respectively, an implantable medical device, such as one of FIG. 1, in accordance with an aspect of this disclosure and one or more embodiments.

### DETAILED DESCRIPTION

The subject matter of this disclosure is described and explained in the following detailed description with reference to the non-limiting aspect(s), embodiment(s), example(s), feature(s), element(s), and step(s), as the case may be, presented herein and illustrated in the accompanying non-limiting drawings and/or figures. Recognizing that these may vary, the skilled addressee shall readily understand that any other variants thereof and any combination of these other variants, as the case may be, are contemplated without departing from the scope of this disclosure, even if they are all not explicitly described herein.

Therefore, these non-limiting aspect(s), embodiment(s), example(s), feature(s), and/or element(s) is/are intended merely to facilitate an understanding of way(s) in which the claimed subject matter may be reduced to practice by the skilled addressee. Accordingly, these shall not to be construed as limiting the scope of the claimed subject matter, which is defined solely by the accompanying claims and applicable law.

The terminology used herein is only for the purpose of describing the claimed subject matter and is not intended to limit the scope hereof. Unless defined otherwise, all technical, engineering, scientific, and other relevant terminology used herein have the same meanings as commonly understood by the skilled addressee.

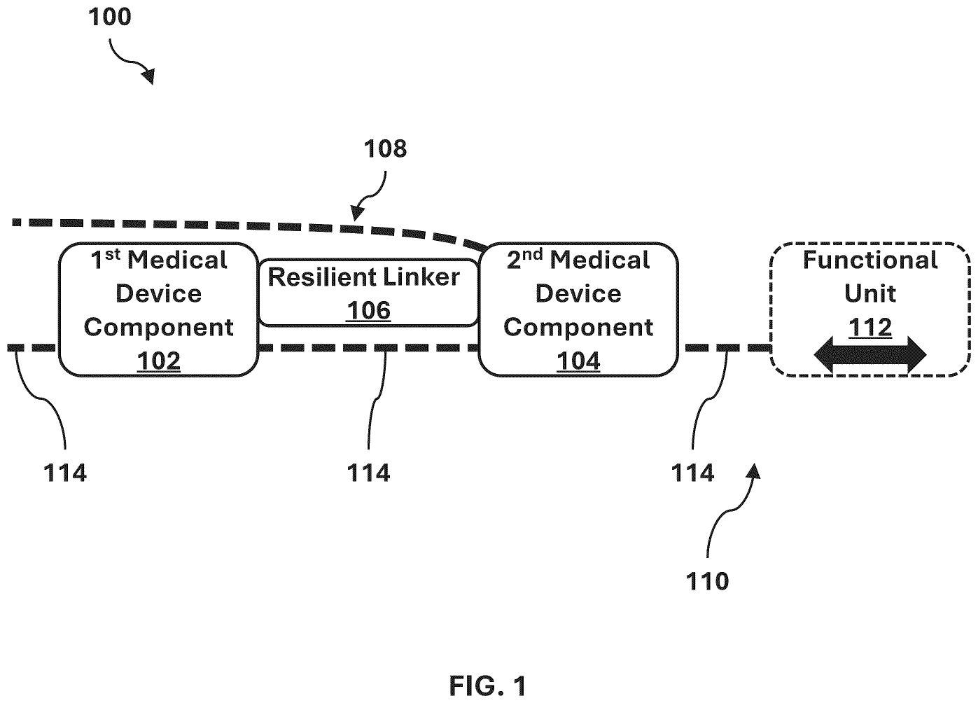

With the foregoing in mind, as schematically represented in FIG. 1, for example, this d isclosure generally relates to an implantable medical device 100 that includes a first medical device component 102, a second medical device component 104, and a linker 106 configured to link the first medical device component 102 and the second medical device component 104 together, according to an aspect of this disclosure. The linker 106 is configured to flex, bend, articulate, pivot, hinge, and/or swivel such that the first medical device component 102 and/or the second medical device component 104 may be positioned for the implantable medical device 100 to be used appropriately in vivo, such as when the implantable medical device 100 is introduced, routed, navigated, delivered, anchored, assembled, operated, and/or implanted in vivo, as well as when the implantable medical device 100 is unassembled, unanchored, retrieved, externalized, and/or explanted in vivo. The implantable medical device 100 may include any transcatheterly and/or percutaneously implantable medical device.

The first medical device component 102 may be configured to run in a subject's body, such as along in a lumen thereof; and the second medical device component 104 may be configured to be anchored in a subject's body and/or may be configured to be electrically or mechanically operated in a subject's body. As such, the first medical device component 102 may be referred to herein as an “elongated medical device”, and the second medical device component 104 may be referred to herein as an “anchorable and/or operable medical device”.

The second medical device component 104 may be electrically or mechanically operated through the linker 106 and/or the first medical device component 102 by a controller operatively connected to the first medical device component 102. Alternatively, the second medical device component 104 optionally include a power lead 108 (represented in dashed lines in FIG. 1 as optional) configured to run along (as represented) or through both the linker 106 and the first medical device component 102, and configured to operatively connect the controller for electrically or mechanically operating the second medical device component 104. For example, the power lead 108 may be an electrical lead configured to electrically operate the second medical device component 104 through it, or may be a driveshaft for mechanically operating the second medical device component 104 through it. The second medical device component 104 may be intracorporeally operated in a subject's body by the controller extracorporeally located outside the subject's body. The second medical device component 104 may also be operated manually by an operator.

Still referring to FIG. 1, the implantable medical device 100 may optionally include an assemblable medical device 110 (represented in dashed lines in FIG. 1 as optional) provided with a functional unit 112 and an elongated control structure 114 that projects therefrom, according to one or more embodiment(s). The implantable medical device 100 may also optionally include multiple assemblable medical devices 110, as described herein. The functional unit 112 is configured to assemble to and unassemble from the second medical device component 104 (as illustrated), the first medical device component 102, and/or the linker 106.

The elongated control structure 114 may be configured to be manipulated or actuated for assembling the functional unit 112 to and for unassembling the functional unit 112 from the second medical device component 104. The elongated control structure 114 may further be configured to be positioned along or through the first medical device component 102, the second medical device component 104, and/or the linker 106. As such, the elongated control structure 114 may be in a slidable relationship with the first medical device component 102, the second medical device component 104, and/or the linker 106 for assembling and unassembling the functional unit 112.

Manipulation or actuation of the elongated control structure 114 moves the functional unit 112 toward the first medical device component 102, the second medical device component 104, and/or the linker 106 for assembling the functional unit 112 to at least one of these. Manipulation or actuation of the elongated control structure 114 moves the functional unit 112 away from the first medical device component 102, the second medical device component 104, and/or the linker 106 for unassembling the functional unit 112 from at least one of these. Such movement of the functional unit 112 is indicated by a double-headed arrow in FIG. 1.

Accordingly, pulling the elongated control structure 114 away from the first medical device component 102 moves the functional unit 112 towards the second medical device component 104, causing the functional unit 112 to assemble thereto. Conversely, pushing the elongated control structure 114 toward the first medical device component 102 moves the functional unit 112 away from the second medical device component 104, causing the functional unit 112 to unassemble therefrom. The assembling and unassembling of the functional unit 112 and the implantable medical device 100 may be commanded from inside or outside the subject's body. For example, the functional unit 112 may be intracorporeally assembled and intracorporeally unassembled in a subject's body by manipulation by an operator or actuation of a portion of the elongated control structure 114 that is extracorporeally disposed outside the subject's body.

Therefore, the assemblable medical device 110 is intracorporeally convertible between an assembled configuration and an unassembled configuration. In the assembled configuration, the functional unit 112 and at least one of the second medical device component 104 (as represented), the first medical device component 102, and/or the linker 106 are assembled together. In the unassembled configuration, the functional unit 112 and at least one of the second medical device component 104 (as represented), the first medical device component 102, and/or the linker 106 are unassembled from each other.

The assemblable medical device 110 may be configured for the functional unit 112 to be electrically or mechanically operated through the elongated control structure 114 by a controller (not shown) operatively connected to the elongated control structure 114. For example, the elongated control structure 114 may be an electrical lead configured to electrically operate the functional unit 112 through it by the controller, or may be a driveshaft configured to mechanically operate the functional unit 112 through it by the controller. The assemblable medical device 110 may also be configured for the functional unit 112 to be manually operated through the elongated control structure 114 by an operator (not shown). The functional unit 112 may be intracorporeally operated, in the assembled and unassembled configurations, in a subject's body by the controller located outside or inside the subject's body.

As described hereinbefore, the linker 106 is flexible (also referred to herein as a “flexible linker 106”), for example by being capable to be bent or modified without breaking and/or by being capable to adapt shape under force. The linker 106 may be also compliant (also referred to herein as a “compliant linker 106”), for example by being capable of yielding or deforming under force. The linker 106 may be also resilient (also referred to herein as a “resilient linker 106”), for example by being able to absorb energy when deformed and return to original shape without rupture or permanent deformation. The linker 106 may be also deformable (also referred to herein as a “deformable linker 106”), for example by being able to capable of undergoing a shape change under force. The linker 106 may be flexible, compliant, resilient, and/or deformable.

Additionally, the linker 106 may be converted or transitioned from a first arrangement toward a second arrangement and be biased or overcomeably biased toward the first arrangement, which may include any linear, arced, angular, curved, and/or folded arrangements. For example, the linker 106 may have one of a general linear arrangement and a comparatively non-linear arrangement (also referred to herein as an “initial arrangement”), be converted or transitioned to the other one of the general linear arrangement and the comparatively non-linear arrangement (also referred to herein as a “subsequent arrangement”), and return by itself to the initial arrangement. The linker 106 may be non-biased and may remain in any given arrangement, for example, in response to a force applied thereto or after a force was applied thereto. For example, the linker 106 may be converted or transitioned from the initial arrangement to the subsequent arrangement and remain in the subsequent arrangement.

The mechanical property(ies) of the linker 106, notably the one(s) relating to the flexible, compliant, resilient, and/or deformable aspect(s) thereof, may be the same as or may be different from the one(s) of the first medical device component 102 and/or the second medical device component 104. For example, the linker 106 may be more flexible than the first medical device component 102 and/or the second medical device component 104. The mechanical properties relating to the flexible, compliant, resilient, and/or deformable aspect(s) of the linker 106 may be adjusted or selected to fit any particular need, such as one(s) for using the implantable medical device 100 in vivo.

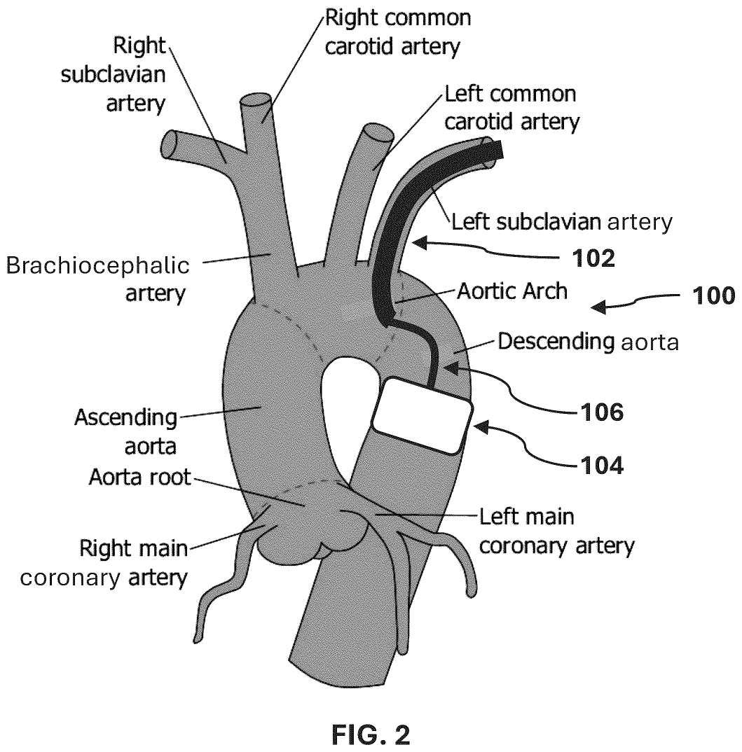

FIGS. 2-5, for example, schematically represent possible implantation modalities of the implantable medical device 100 in a subject's body, such as in the vascular system thereof, according to an aspect of this disclosure.

Referring to FIG. 2, the implantable medical device 100 is represented implanted with the first medical device component 102 provided as an elongated medical device at least partially located in the left subclavian artery and the aortic arch, and with the second medical device component 104 provided as an anchorable and/or operable medical device optionally anchored in the aorta and/or optionally operated therein, according to one or more embodiment(s). As illustrated, a flexible linker 106 enables the second medical device component 104 to be appropriately positioned, anchored, and/or operated in the descending aorta. The flexible linker 106 may also enable the second medical device component 104 to be appropriately positioned, anchored, and/or operated in the descending aorta, as illustrated in FIG. 2, but also in the ascending aorta and/or the aortic arch.

Advantageously, the flexible linker 106 enables the implantable medical device 100 to appropriately bend during routing or navigation in a subject's body, such as through tortuous anatomies, and/or to position appropriately the first and/or second medical device component(s) 102, 104 at a same implantation site or at their respective implantation sites.

The position of the first and/or second medical device component(s) 102, 104 may impact the operation thereof in the subject's body. For example, if mispositioned, such as when in contact with a lumen wall, the electrical power consumption of any one of the first and second medical device components 102, 104 may increase relative to when they are appropriately positioned away from the lumen wall. A mispositioning may also cause unwanted interaction of the first and second medical device component(s) 102, 104 with a lumen wall. For example, when any one of the first and second medical device components 102, 104 is/are blood pump(s), such interaction may cause the blood pump to suction the lumen wall instead of blood, impairing its intended function.

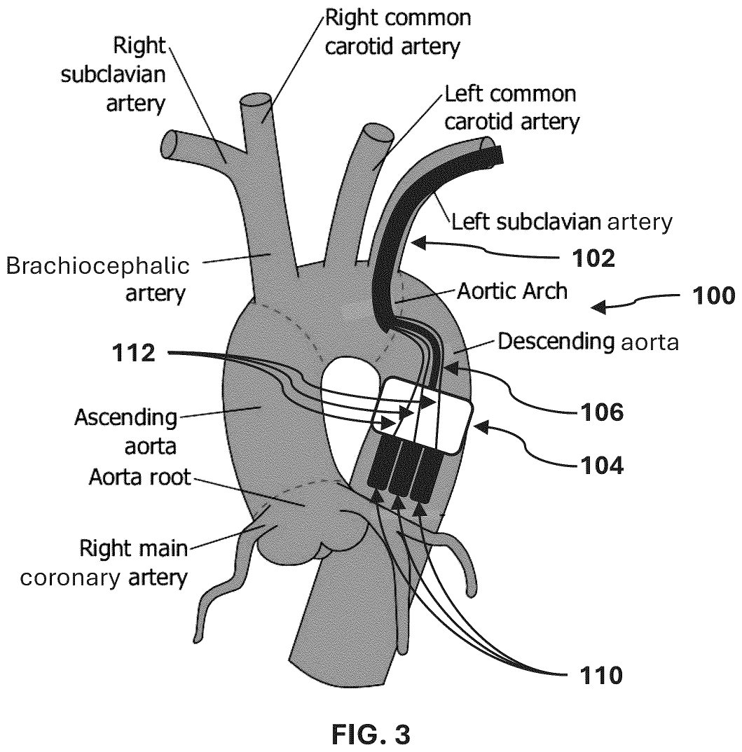

When the first medical device component 102 is provided as an elongated medical device, such as a cannula or a catheter, the flexible linker 106 may be required to be more flexible than the first medical device component 102 for the second medical device component 104 to be positioned appropriately and/or operated appropriately in a subject's body. This may be the case, as illustrated in FIGS. 2-3, with the first medical device component 102 extending along the left subclavian artery, the second medical device component 104 positioned and optionally anchored in the aorta in a suprarenal position, and the flexible linker 106 accommodating the tortuosity of the aorta (as illustrated) and/or of the left subclavian artery.

Further, since the flexible linker 106 is more flexible than the first medical device component 102, the stiffness of the first medical device component 102 may be adjusted or selected to facilitate navigation of the first medical device component 102 in a subject's body. For example, an appropriate stiffness of a first medical device component 102 may facilitate the pushing and advancing the first medical device component 102 along a subject's vascular system.

Absent the flexible linker 106 and with the first medical device component 102 attached to the second medical device component 104, the second medical device component 104 would be mispositioned and/or misaligned relative to the aorta, hindering or preventing the appropriate positioning, anchoring, operation, and /r functioning of the second medical device component 104, as the case may be. Such mispositioning and/or misaligning may happen, for example, when the elongated medical device is not compliant, resilient, flexible, and/or bendable enough to appropriately accommodate a lumen of one or more tortuous body conduits or a lumen of one or more body conduits that have one or more acute angles.

Referring to FIG. 3, the implantable medical device 100 of FIG. 2 is represented but with the second medical device component 104 assembled to three assemblable medical devices 110 and optionally anchored in the aorta, according to one or more embodiment(s). Also, each assemblable medical device 110 may be operated in the aorta. The three assemblable medical devices 110 may also be anchored and/or operated in the in the descending aorta, as illustrated in FIG. 3, but also in the ascending aorta and/or the aortic arch. Similarly to the flexible linker 106, the elongated control structures 114 of the assemblable medical devices 110 are configured to flex and/or bend.

The appropriate positioning and/or alignment of the second medical device component 104 relative to the aorta advantageously enable the assemblable medical devices 110 to be assembled to the second medical device component 104, unassembled from the second medical device component 104, and operated in the aorta. For example, such positioning and/or alignment enable the downstream-oriented end portion of the assemblable medical devices 110 to be snared downstream from the aorta for the assemblable medical devices 110 to be unassembled from the second medical device component 104, as described herein.

Absent the flexible linker 106, the downstream-oriented end portion of the assemblable medical devices 110 would abut the lumen wall, hindering or preventing appropriate snaring, operation, and/or functioning of the assemblable medical devices 110, as the case may be.

Referring to FIGS. 2-3, the first medical device component 102 may be referred to herein as an “intra-cardiac device”, and the second medical device component 104 may be referred to herein as an “intra-aortic device”.

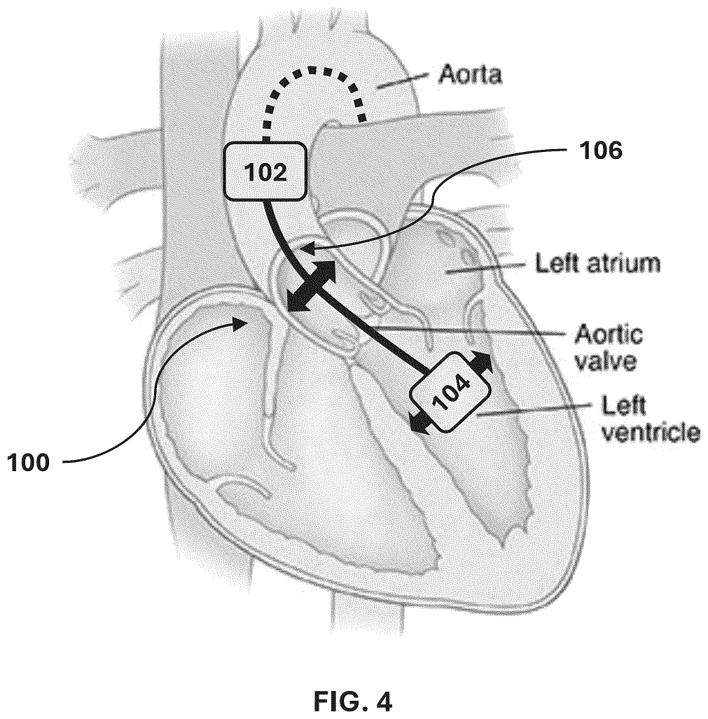

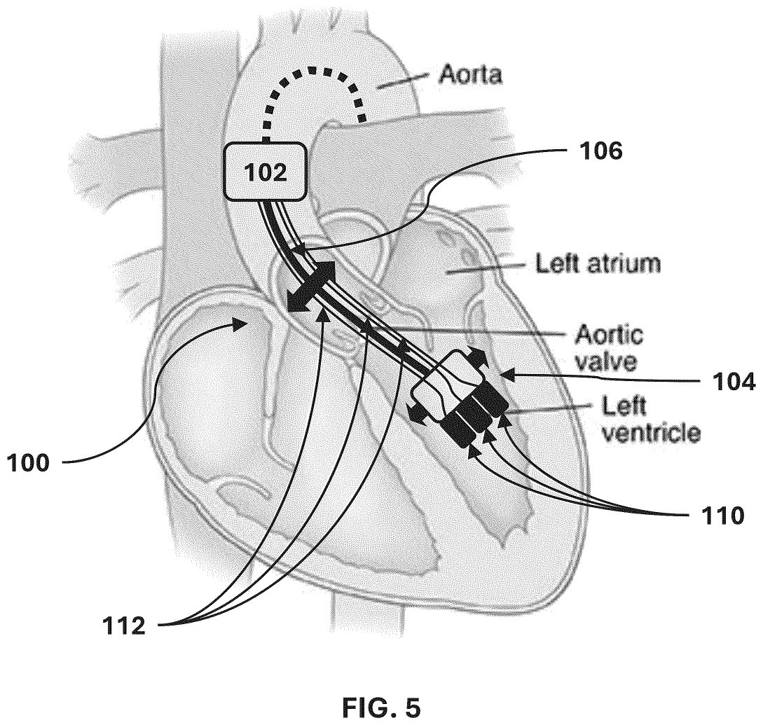

Referring to FIG. 4, the implantable medical device 100 is represented implanted with the first medical device component 102 optionally anchored in the aorta and optionally including an elongated portion (represented in dashed lines in FIG. 4-5 as optional) configured to run therefrom, with the second medical device component 104 optionally anchored in the aorta and/or optionally operated therein, and finally with the flexible linker 106 positioned through the aortic valve, according to one or more embodiment(s).

As such, the flexible linker 106 advantageously enables the first medical device component 102 and/or the second medical device component 104 to be appropriately positioned and/or anchored in their respective lumens, and/or the implantable medical device 100 to appropriately move (indicated by double-headed arrows in FIGS. 4-5) for accommodating the movement of the left ventricle of a beating heart.

Absent the flexible linker 106 and with the first medical device component 102 attached to the second medical device component 104, the first medical device component 102 and/or the second medical device component 104 would be mispositioned and/or misaligned relative to their respective lumens, hindering or preventing the appropriate operation or functioning of the first medical device component 102 and/or the second medical device component 104, as the case may be. Such mispositioning and/or misaligning may happen, for example, when the first medical device component 102 or the second medical device component 104, but not the flexible linker 106, is positioned through the aortic valve, such that the movement of the left ventricle cannot be appropriately accommodated.

Referring to FIG. 5, the implantable medical device 100 of FIG. 4 is represented but with the second medical device component 104 assembled to three assemblable medical devices 110 and optionally anchored in the left ventricle, according to one or more embodiment(s). Also, each assemblable medical device 110 may be operated in the left ventricle. Similarly to the flexible linker 106, the elongated control structures 114 of the assemblable medical devices 110 are positioned through the aortic valve and are configured to appropriately move for accommodating the movement of the left ventricle.

The appropriate positioning and/or alignment of the first medical device component 102 and/or the second medical device component 104 relative to their respective lumens advantageously enable the assemblable medical devices 110 to be assembled to the second medical device component 104, unassembled from the second medical device component 104, and operated in the left ventricle.

Referring to FIGS. 4-5, the first medical device component 102 may be referred to herein as an “intra-aortic device”, and the second medical device component 104 may be referred to herein as an “intra-cardiac device”.

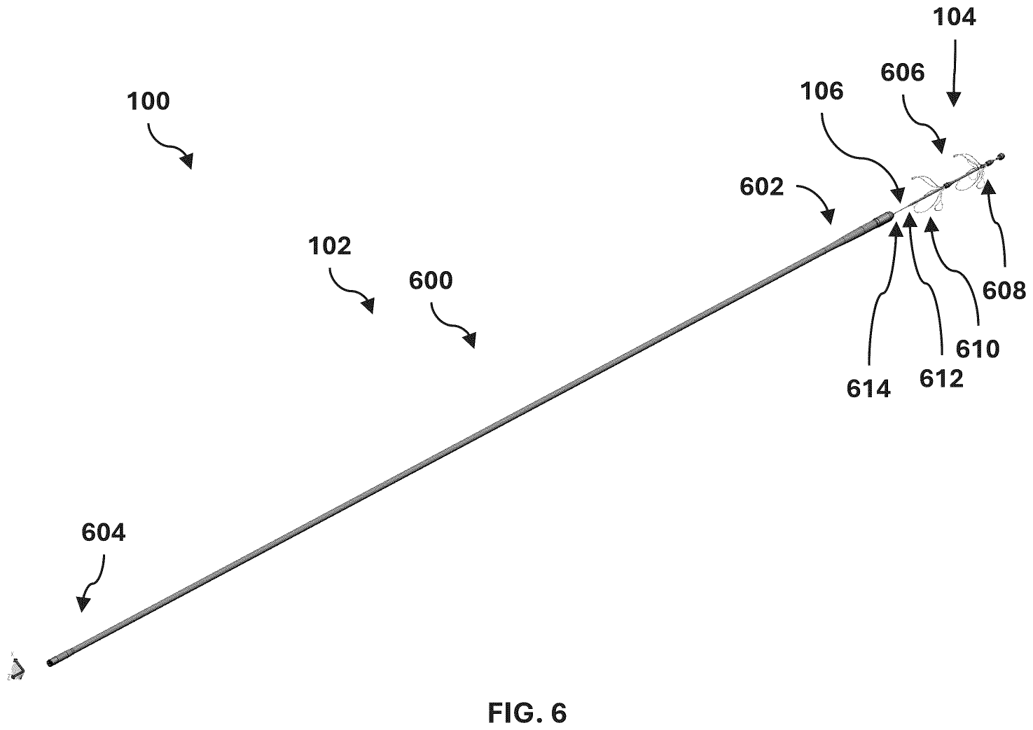

FIGS. 6-22 illustrate the implantable medical device 100 that includes the first medical device component 102 optionally provided as an elongated medical device, such as a cannula or a catheter 600; the second medical device component 104 optionally provided as various anchorable medical devices; and the flexible linker 106, according to one or more embodiment(s).

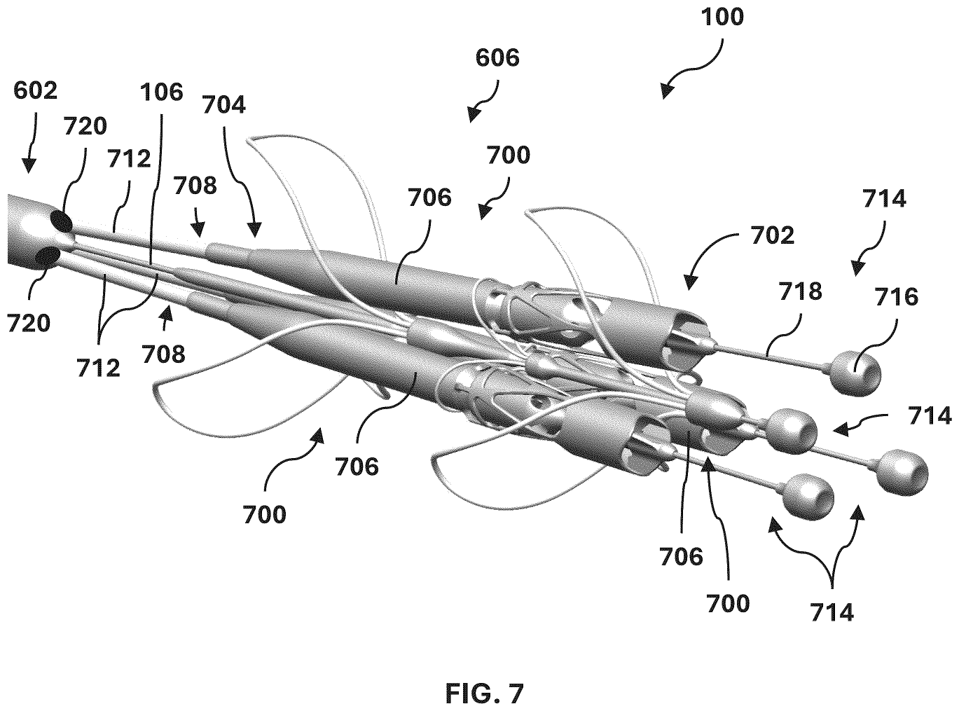

In particular, FIGS. 6-7 illustrate the catheter 600 having first and second end portions 602, 604; an anchorable medical device optionally provided as an anchor 606 having first and second end portions 608, 610; and the flexible linker 106 having a first end portion 612 configured to directly or indirectly couple the second end portion 610 of the anchor 606, and having a second end portion 614 configured to directly or indirectly couple the first end portion 602 of the catheter 600, according to one or more embodiment(s). No assemblable medical device is shown in FIG. 6 for the sake of clarity.

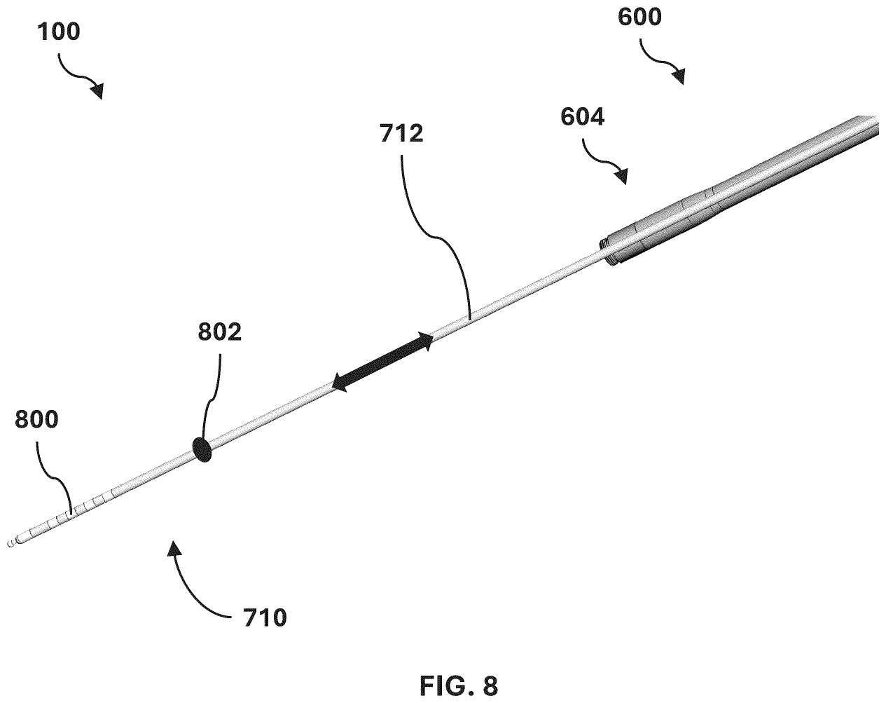

The implantable medical device 100 may include one or more assemblable medical devices 110. For example, as illustrated in FIGS. 7-8, the implantable medical device 100 optionally includes three optional assemblable medical devices 110 provided as three assemblable pumps 700 that are assembled to the anchor 606, according to one or more embodiment(s). As such, the functional unit 112 of the assemblable medical devices 110 has first and second end portions 702, 704 and is provided as a pump unit 706. The elongated control structure 114 of the assemblable medical devices 110 has first and second end portions 708, 710 and is provided as an electrical lead 712. The second end portion 704 of the pump unit 706 is configured to directly or indirectly couple the first end portion 708 of the electrical lead 712. As illustrated in FIG. 8, the second end portion 710 of the electrical lead 712 is provided with an electrical connector 800 configured to electrically connect a controller for operating the pump unit 706, according to one or more embodiment(s).

Each pump unit 706 optionally include an impeller configured to impart movement to a fluid, an electric motor configured to drive the impeller, a magnetic coupling configured to magnetically couple the impeller and the electric motor together for the electric motor to drive the impeller, and a casing configured to enclose the electric motor and from which the electrical lead 712 projects therefrom.

Alternatively, the functional unit 112 may be void of an electric motor and be configured to be mechanically operated by the elongated control structure 114 provided as a driveshaft (not shown).

The functional unit 112 may include a capturable element configured to be intracorporeally captured by a medical tool, such as in the vascular system. The capturable element may also be configured not to intracorporeally engage with or attach to the second medical device component 104 since this may prevent or may interfere with the assembly and unassembly of the assemblable medical device 110. For example, still referring to FIG. 7, each pump unit 706 optionally includes a respective capturable element 714 configured to be intracorporeally captured by a medical tool, such as a snare, in a subject's body. The capturable element 714 projects generally axially from the pump unit 706, such as from the shroud thereof. The capturable element 714 may also project at an angle from the pump unit 706. The capturable element 714 includes a protrusion 716 and a stem 718 that attaches the protrusion 716 to the pump unit 706. The protrusion 716 may include a magnet or an electromagnet, or be magnetizable for magnetic interaction with a medical tool, such as a snare, used to capture the capturable element 714. The stem 718 may have the same mechanical properties as the flexible linker 106.

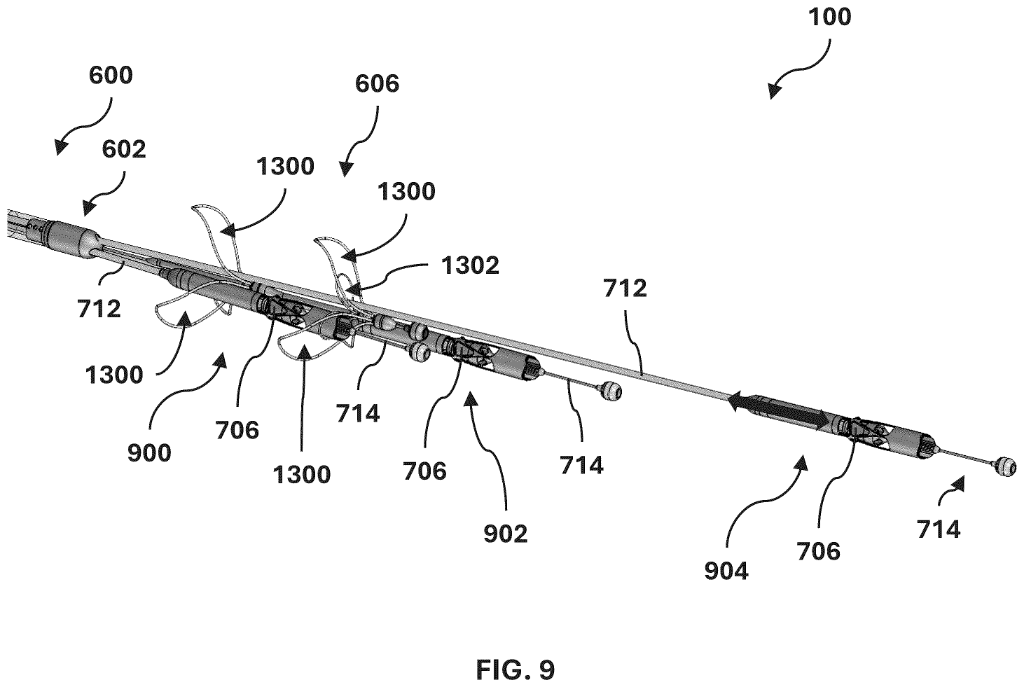

The elongated control structure 114 may be manipulable for assembling the functional unit 112 to and unassembling the functional unit 112 from the second medical device component 104. For example, referring back to FIGS. 8-9, the second end portion 710 of each electrical lead 712 can be pulled away from the second end portion 604 of the catheter 600 and can also be pushed toward the second end portion 604 of the catheter 600 for moving a respective pump unit 706 toward and away from the anchor 606, respectively, according to one or more embodiment(s). Both pulling and pushing are represented in FIG. 8 by a double-headed arrow, while corresponding movements of the respective pump unit 706 are represented in FIG. 9 by a double-headed arrow. Pulling the electrical lead 712 causes the pump unit 706 to assemble to the anchor 606, while pushing the electrical lead 712 causes the pump unit 706 to unassemble from the anchor 606. As such, the assemblable medical devices 110 are in a slidable relationship with at least one of the catheter 600, the anchor 606, and the flexible linker 106.

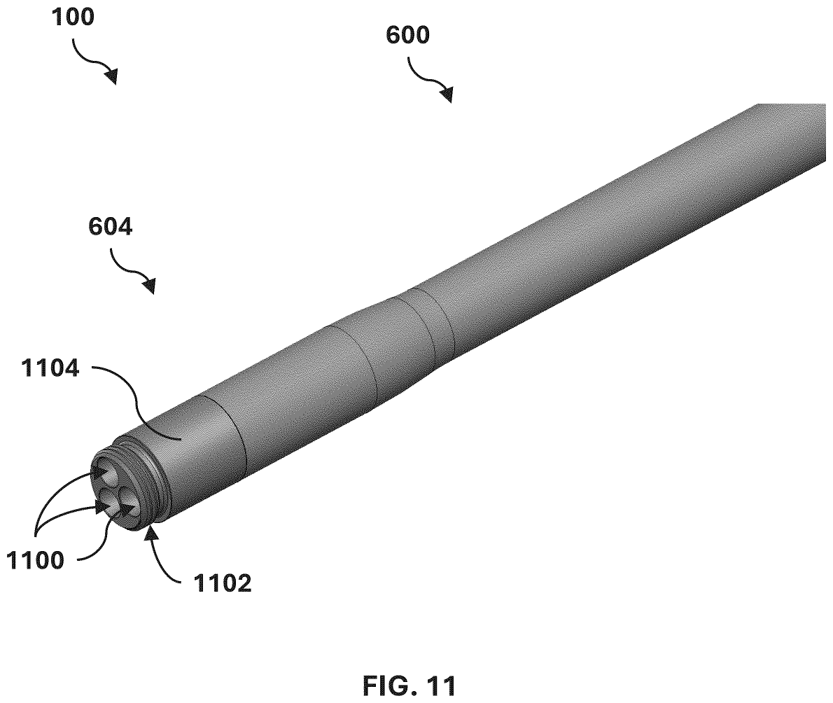

It is to be noted for the purpose of this explanation that the electrical lead 712 is shown superposed to the catheter 600 in FIG. 8 instead of being slidably received in a longitudinal guide channel 1100 (shown in FIG. 11) of the catheter 600 as in FIG. 9.

The three functional units 706 may be simultaneously assembled and unassembled by simultaneously pulling and pushing, respectively, the corresponding electrical leads 712. The three functional units 706 may also be consecutively assembled and unassembled by consecutively pulling and pushing, respectively, the corresponding electrical leads 712.

FIG. 9 illustrates an assemblable pump 900 in the assembled configuration with the pump unit 706 thereof assembled to the anchor 606, an assemblable pump 902 in a partially assembled/unassembled configuration with the pump unit 706 thereof partially assembled to or partially unassembled from the anchor 606, and the assemblable pump 904 in the unassembled configuration with the pump unit 706 unassembled from the anchor 606, according to one or more embodiment(s). Each of the assemblable pumps 900, 902, 904 is the same than the assemblable pump(s) 700.

Assembled to the anchor 606, the assemblable medical devices 110 may be arranged in various ways. For example, the three pump units 706 may be generally arranged in parallel and generally non-coaxially relative to each other and/or relative to at least one of the anchor 606 (e.g., as illustrated referring back to FIG. 7), the catheter 600, and the flexible linker 106. The three pump units 706 may also be transversally arranged side to side relative to each other (e.g., as illustrated referring back to FIG. 7). Alternatively, when a single assemblable medical device 110 is provided to the implantable medical device 100, its functional unit 112 may or may not be arranged coaxially and/or in parallel relative to at least one the first medical device component 102, the second medical device component 104, and the flexible linker 106.

Unassembled from the anchor 606, the assemblable medical devices 110 may also be arranged in various ways. For example, one or more pump units 706 may be arranged substantially parallel relative to at least one of the first medical device component 102, the second medical device component 104, and the flexible linker 106. The three functional units 706 may also be longitudinally arranged one after the other. Alternatively or additionally, one or more pump units 706 may be arranged coaxially relative to at least one of the first medical device component 102, the second medical device component 104, and the flexible linker 106.

Each assemblable pump 700 may be intracorporeally operated in the assembled, partially assembled/unassembled, or unassembled configuration in a subject's body, such as in the vascular system thereof, by a controller (not shown) extracorporeally disposed outside the subject's body.

The assemblable medical device 110 may be a blood pump, such as an axial-flow blood pump or a mixed-flow blood pump.

Referring back to FIG. 6, the catheter 600 includes an elongated body 616 that extends between the first and second end portions 602, 604, and defines one or more longitudinal guide channels 1100 (shown in FIG. 11) at least partially extending between the first and second end portions 602, 604, according to one or more embodiment(s). The longitudinal guide channel(s) 1100 is/are configured to slidably receive one or more electrical lead(s) 712 therein for assembling and unassembling corresponding pump unit(s) 706, as described. Alternatively, the first medical device component 102 may be provided with one or more guide holes, such as when the first medical device component 102 does not have an elongated body. Still alternatively, one or more electrical lead(s) 712 may be configured to be positioned outside along the catheter 600.

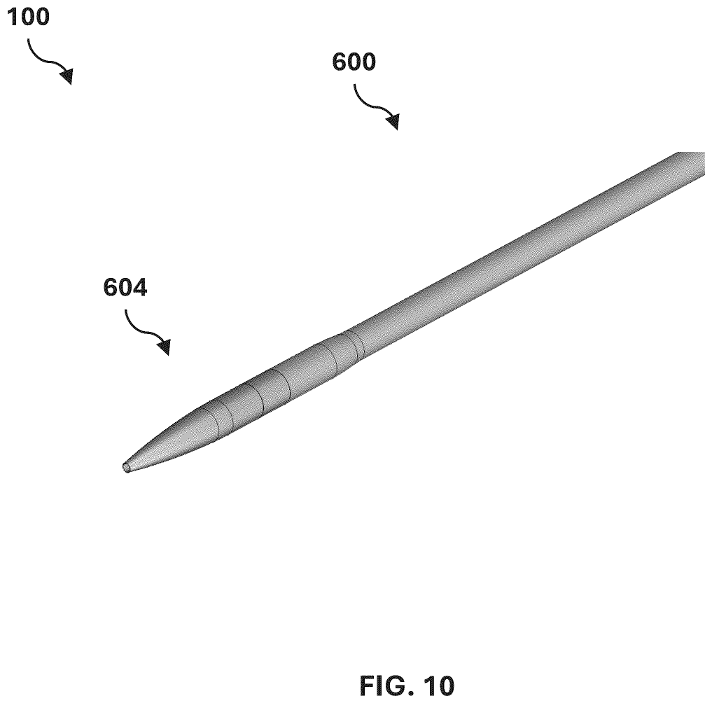

The catheter 600 may be configured to be atraumatic when used in transcatheter applications, such as when the second end portion 604 of the catheter 600 is introduced through a first subject's body intracorporeal access, routed in a subject's body, such as in a lumen of a subject's body conduit like the vascular system, and externalized through a second subject's body intracorporeal access. For example, as illustrated in FIG. 10, the second end portion 604 of the catheter 600 is optionally of a rounded shape, such as of a tapered and/or profiled shape (in this case, also referred to herein as an “atraumatic end portion or tip”), similar to a dilator tip of an introducer sheath, according to one or more embodiment(s). Also, the second end portion 604 of the catheter 600 may optionally include a guidewire (not shown) that projects from the most tapered and/or profiled portion thereof and configured to be intracorporeally routed in a subject's body, such as in the vascular system.

The second end portion 604 of the catheter 600 may be configured to removably attach (in this case, also referred to herein as a “removable atraumatic end portion or tip”) to the remainder of the catheter 600 (in this case, also referred to herein as the “body of the catheter”), such that one or more longitudinal guide channel(s) 1100 (best shown in FIG. 11) and/or electrical lead(s) 712 slidably received therein is/are protected from the outside environment when the second end portion 604 is attached. The longitudinal guide channel(s) 1100 and/or electrical lead(s) 712, when present, is/are accessible to an operator when the second end portion 604 is removed. For example, as illustrated in FIG. 11, an optional screw thread(s) 1102 is provided to the body of the catheter 600 and is configured to engage a corresponding screw thread(s) (not shown) provided to the removable atraumatic end tip of the catheter 600 of FIG. 10, according to one or more embodiment(s). Alternatively, the second end portion 604 of the catheter 600 may be configured to removably attach the body of the catheter 600 by interference fit or any other suitable attachment means. It is to be noted that the removable second end portion 610 of the catheter 600 is omitted from FIGS. 6, 8, and 11.

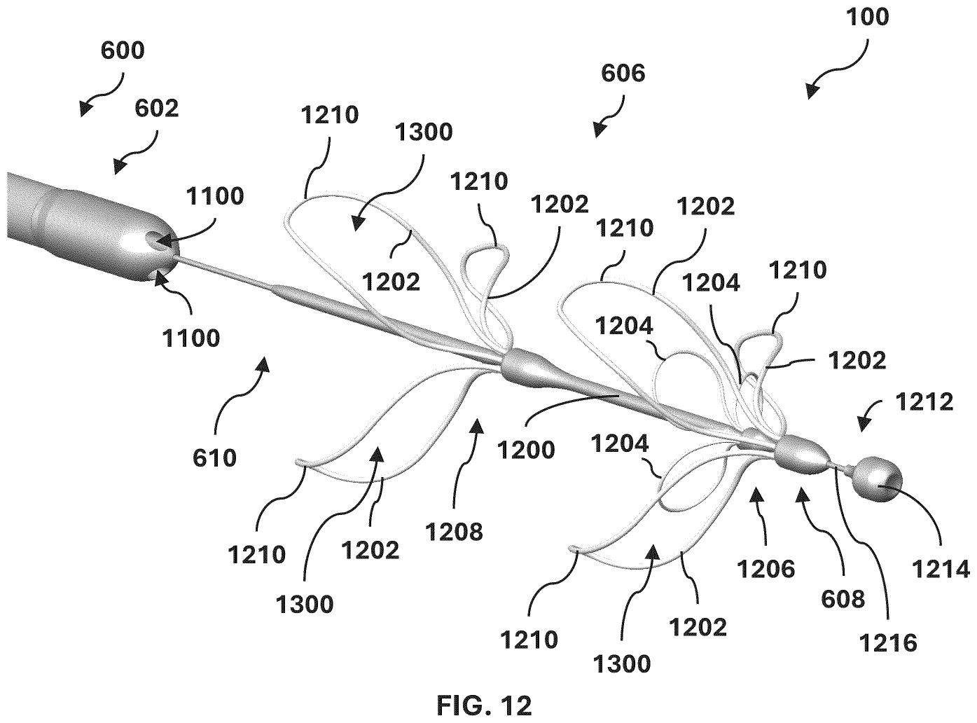

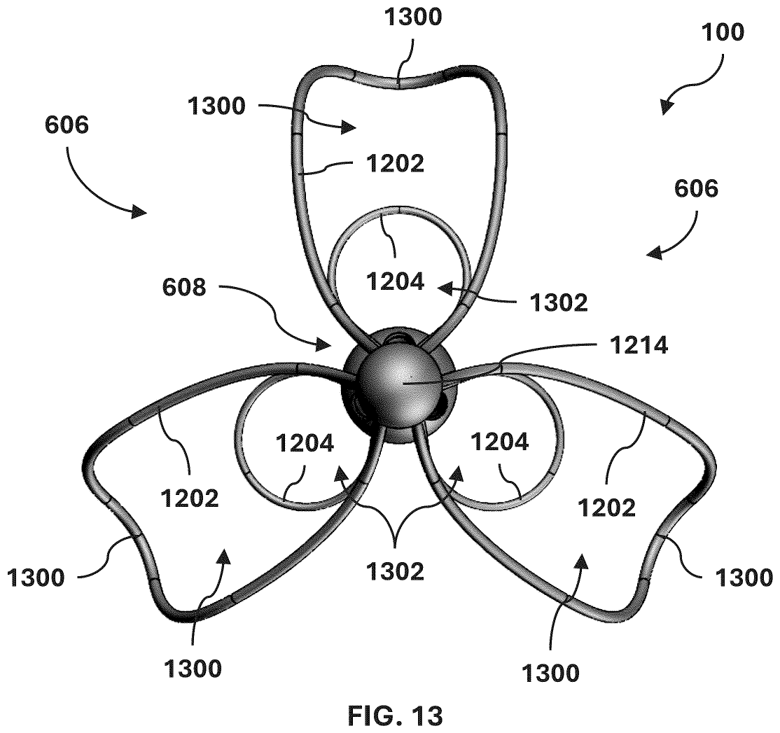

FIGS. 12-13 illustrate the anchor 606 that includes a post 1200 extending between the first and second end portions 608, 610 of the anchor 606; six anchoring loops 1202 (also referred to herein as “anchoring elements”) configured to intracorporeally anchor the anchor 606 in a subject's body; and three assembling loops 1204 (also referred to herein as “assembling loops”) each configured to intracorporeally assemble a respective assemblable pumps 700 in a subject's body, according to one or more embodiment(s). The second end portion 610 of the anchor 606 is directly or indirectly coupled to the first end portion 612 of the flexible linker 106, resulting in the post 1200 axially projecting from the flexible linker 106. Alternatively, the anchor 606 may include any number of anchoring loops 1202 and assembling loops 1204, and may project from the flexible linker 106 in any direction.

The post 1200 may be inflexible, less flexible than the flexible linker 106, as flexible as the flexible linker 106, or more flexible than the flexible linker 106. When as flexible as the flexible linker 106, the flexible linker 106 may be the post 1200. For example, the post 1200 may be made of nitinol, stainless steel, titanium, any biocompatible alloys, any biocompatible polymers, and the like.

As illustrated in FIGS. 12-13, the six anchoring loops 1202 project generally radially from the post 1200, with three anchoring loops 1202 grouped in a first cluster 1206 positioned proximate the first end portion 608 of the anchor 606, and three other anchoring loops 1202 grouped in a second cluster 1208 positioned at an intermediate portion of the anchor 606, according to one or more embodiment(s). The first and second clusters 1206, 1208 are axially spaced apart from each other along the post 1200. Alternatively, the second cluster 1208 may be positioned proximate the second end portion 610 of the anchor 606. Also, any number of anchoring loops 1202 may be grouped in any number of clusters.

Two or more clusters of anchoring loop(s) 1202 better maintain the post 1200 in a coaxial alignment with the lumen, as opposed to a single cluster of anchoring loops 1202 which is more prone to coaxial misalignment induced by movement of the catheter 600 in the lumen. Coaxial misalignment of the anchor 606 may traumatize or may damage the lumen wall, and/or may cause the functional unit 112 assembled to the anchor 606 to contact or to abut the lumen wall, possibly traumatizing or damaging the lumen wall as well.

The anchoring loops 1202 may be configured to exert pressure to a lumen wall for engaging the lumen wall in a way that the anchor 606 may be moved therealong without traumatizing or damaging the lumen wall. In particular, each anchoring loop 1202 has a respective engagement portion 1210 configured to atraumatically engage an anatomical structure, such as a lumen wall, for anchorage. With the engagement portion 1210 so engaged to a lumen wall, the anchor 606 may be moved in a lumen of a subject's body conduit, such as the vascular system, by pulling and/or pushing the catheter 600 without traumatizing or damaging the lumen wall. For example, referring to FIG. 13, the engagement portion 1210 is provided as an arched portion 1300 oriented toward the second end portion 610 of the anchor 606, according to one or more embodiment(s). Alternatively or additionally, the arched portion 1300 may be oriented toward the first end portion 608 of the anchor 606. Still alternatively, the anchoring loops 1202 may be configured to engage the lumen wall in a way that the anchor 606 cannot be moved without traumatizing or damaging the lumen wall.

Referring back to FIG. 9 and as also illustrated in FIGS. 12-13, each anchoring loop 1202 includes an optional respective opening 1302 configured to slidably receive a respective electrical lead 712 and optionally a respective pump unit 706 therethrough, according to one or more embodiment(s).

As illustrated in FIG. 12, the three assembling loops 1204 project generally radially from the post 1200 and grouped in a single cluster positioned proximate the first end portion 608 of the anchor 606, adjacent the first cluster 1206, according to one or more embodiment(s). Alternatively, the single cluster may be positioned proximate the second end portion 610 of the anchor 606, or the assembling loop(s) 1204 may be grouped in two clusters, generally similarly to the anchoring loop(s) 1202. Also, any number of assembling loops 1204 may be grouped in any number of clusters.

As illustrated in FIGS. 7, 9, 12, and 13, each assembling loop 1204 (also referred to herein as an “assembling element”; best shown in FIGS. 9 and 13) forms a respective opening 1302 configured to slidably receive a respective electrical lead 712 therethrough and further configured to slidably receive and/or slidably engage a respective pump unit 706 therein, according to one or more embodiment(s). Alternatively, each assembling loop 1204 may form multiple openings 1302.

In particular, as illustrated in FIG. 9, the electrical lead 712 of the assemblable pump 904 is received through or a respective opening 1302, the pump unit 706 of the assemblable pump 902 is partially received or engaged through a respective opening 1302 (not clearly visible in FIG. 9), and the pump unit 706 of the assemblable pump 900 is received or engaged through a respective opening 1302 (not clearly visible in FIG. 9), according to one or more embodiment(s). Also, as illustrated in FIG. 7, each functional unit 112 of three assemblable pumps 700 are received or engaged in a respective opening 1302, according to one or more embodiment(s). So slidably received and/or slidably engaged, each pump unit 706 may be free-floating or may be immobilized.

Referring to FIG. 11, the catheter 600 optionally includes a securing mechanism 1104 configured to secure an electrical lead 712 (not shown) for retaining a corresponding pump units 706 (not shown) in the opening 1302 of the assembling loops 1204, according to one or more embodiment(s). As illustrated, the securing mechanism 1104 is provided to the second end portion 604 of the catheter 600; however, the catheter 600 may be provided anywhere on the first medical device component 102. The assemblable pumps 700 may be retained in the openings 1302 offset relative to each other.

Referring back to FIG. 7, each electrical lead 712 may include, at its first end portion 708, a stop or abutment element 720 configured to abut against or engage with the first end portion 602 of the catheter 600, according to one or more embodiment(s). Additionally or alternatively, referring to FIG. 8, each electrical lead 712 may include, at its second end portion 710, a stop or abutment element 802 configured to abut against or engage with the second end portion 604 of the catheter 600, according to one or more embodiment(s). Although not necessarily shown as such, each electrical lead 712 may be provided with a stop or abutment element 720 and/or a stop or abutment element 802. Also, the stop or abutment element 720 and/or the stop or abutment element 802 may be located at offset locations relative to each other among multiple electrical leads 712.

The stop or abutment elements 720 allow to predetermine the axial position of the functional units 706 in the openings 1302 of the assembling loops 1204 when abutted or engaged. Stop or abutment elements 720 located at offset locations enable the corresponding functional units 706 to be axially offset relative to each other when assembled to the anchor 606. The stop or abutment elements 802 allow to predetermine the axial position and distance of the functional units 706 relative to the first end portion 602 of the catheter 600. Stop or abutment elements 802 located at offset locations enable the corresponding functional units 706 to be axially positioned and axially distanced offset relative to each other when unassembled from assembled to the anchor 606. As such, the stop or abutment element 802 may prevent the second end portion 710 of the electrical leads 712 from entering inside the longitudinal guide channels 1100 when abutted or engaged. This may be useful when the implantable medical device 100 is routed or navigated in a subject's body.

As illustrated in FIGS. 12-13, each assembling loop 1204 generally coincide with a respective anchoring loop 1202, such that an opening 1302 of an assembling loop 1204 and an opening 1302 of the anchoring loop 1202 may slidably receive a respective electrical lead 712 (not shown) and a respective pump unit 706 (not shown) therethrough, according to one or more embodiment(s). Alternatively, the assembling loops 1204 and anchoring loops 1202 may be offset relative to each on the circumference of the post 1200, such as each assembling loop 1204 may be positioned between two anchoring loops 1202. Also, one or more of the assembling loops 1204 may be axially positioned anywhere along the post 1200, such as closer to the first end portion 608 than the anchoring loops 1202, at an intermediate portion of the post 1200, and/or proximate the second end portion 610 of the post 1200.

It will be appreciated that, depending on the axial positioning of the assembling loop(s) 1204 relative to anchoring loop(s) 1202 along the post 1200, an electrical lead 712 and optionally a pump unit 706 of an assemblable pump 700 may be received through the opening 1302 of the assembling loop(s) 1204.

Both the anchoring loops 1202 and the assembling loops 1204 have an expanded configuration, as illustrated in FIGS. 6, 7, 9, 12, and 13, and a non-expanded configuration, according to one or more embodiment(s).

In the expanded configuration, the anchor 606 is configured to intracorporeally anchor itself and to intracorporeally assemble to the assemblable pump 900, such as in the lumen of a subject's body conduit. The anchoring loops 1202 and the assembling loops 1204 generally radially project from the post 1200 (also referred to herein as a “deployed configuration”). The anchoring loops 1202 are configured to engage an anatomical structure, such as a lumen wall. The assembling loops 1204 are configured to assemble to corresponding functional units 706 of the assemblable medical devices 110.

In the non-expanded configuration, the anchor 606 is configured for intracorporeal delivery of the implantable medical device 100. The anchoring loops 1202 and the assembling loops 1204 are generally axially positioned along the post 1200 (also referred to herein as an “undeployed configuration”). So positioned, the anchoring loops 1202 and/or the assembling loops 1204 may project toward the first end portion 608 or the second end portion 610 of the anchor 606.

When made of a shape-memory material and overcomeably biased toward the expanded configuration, the anchoring loops 1202 and the assembling loops 1204 may be converted from the non-expanded configuration to the expanded configuration by causing the anchor 606 to exit from a sheath, and be converted from the expanded configuration to the non-expanded configuration by introducing the anchor 606 in a sheath. As such, the anchor 606 is self-expandable. When made of a non-shape-memory material, the anchoring loops 1202 and the assembling loops 1204 may be converted from the non-expanded configuration to the expanded configuration by inflating a balloon catheter appropriately positioned relative to the anchor 606. The anchoring loops 1202 and the assembling loops 1204 may be converted between the non-expanded configuration and the expanded configuration, and vice versa, in a lumen of a subject's body conduit, such as in the vascular system.

As illustrated in FIG. 7, the assemblable pumps 700 assembled to the anchor 606 in the expanded configuration are partially surrounded by the first and second clusters 1206, 1208 of the anchoring loops 1202, according to one or more embodiment(s). Such arrangement generally prevents or mitigates any undesired contact of the assemblable pumps 700 with the lumen wall that may possibly traumatize or damage the lumen wall, notably due to the operation of the assemblable pumps 700.

Alternatively to the anchoring loops 1202, the anchoring elements may be provided as anchorable strut(s) (not shown) clustered like the anchoring loop(s) 1202. Each anchorable strut may include a respective engagement portion 1210, which may be provided as a curvature of a most distant end portion of the anchorable strut, opposed to the end portion thereof attached to the post 1200. The anchoring elements may also be provided as a generally rounded geometrical shape at the most distant end portion of the anchorable strut. Each anchorable strut may include an opening, similar to the openings 1302 of the anchoring loops 1202.

The second medical device component 104 may include a capturable element configured to be intracorporeally captured by a medical tool, such as in the vascular system. The capturable element may also be configured not to intracorporeally engage with or attach to the assemblable medical device 110 since this may prevent or may interfere with the assembly and unassembly of the assemblable medical device 110. For example, referring to FIG. 12, the anchor 606 optionally includes a capturable element 1212 configured to be intracorporeally captured by a medical tool, such as a snare, in a subject's body, according to one or more embodiment(s). The capturable element 1212 projects generally axially from the first end portion 608 of the post 1200. The capturable element 1212 may also project at an angle from the post 1200. The capturable element 1212 includes a protrusion 1214 and a stem 1216 that attaches the protrusion 1214 to the first end portion 608 of the post 1200. The protrusion 1214 may include a magnet or an electromagnet, or be magnetizable for magnetic interaction with a medical tool, such as a snare, used to capture the capturable element 1212. The stem 1216 may be inflexible or flexible like the flexible linker 106. The capturable element 1212 is configured to be captured in a lumen of a subject's body conduit, such as in the vascular system.

The flexible linker 106 includes any structures capable of appropriately accommodating a given angle of an anatomical structure, such as in a lumen of a subject's body conduit, in the context of using the implantable medical device 100 in vivo. In such anatomical structure, the implantable medical device 100 not provided with the flexible linker 106 and with the first medical device component 102 directly or indirectly coupled to the second medical device component 104 would be incapable of appropriately accommodating the given angle. The flexible linker 106 may have one or more properties in terms of resilience, compliance, flexibility, and/or bendability that enables the implantable medical device 100 to be appropriately used in a subject's body, such as in the vascular system. This includes but is not limited to introducing, routing, navigating, delivering, anchoring, assembling, operating, and/or implanting in vivo, as well as unassembling, unanchoring, retrieving, externalizing, and/or explanting in vivo. Such implantable medical device may include, for example, an implantable medical device 100 that is configured for transcatheter and/or percutaneous implantation and/or explantation.

Depending on the implantable medical device 100, the flexible linker 106 may have resilience, compliance, flexibility, and/or bendability properties that is lower than, equal to, or superior to the first medical device component 102 and/or the first medical device component 102.

Still for example, the flexible linker 106 may be provided as an articulated joint, a flexible joint, a pivot point, a hinge, a swivel, and the like, as well as any combination(s) thereof.

The flexible linker 106 may have any shapes. For example, the flexible linker 106 may have a generally linear arrangement or an arced arrangement between the first end portion 612 and the second end portion 614 thereof. The flexible linker 106 may define one or more angles between the first end portion 612 and the second end portion 614 thereof. The flexible linker 106 may include one or more folds between the first end portion 612 and the second end portion 614 thereof. The flexible linker 106 may have any combinations of a linear arrangement, one or more angles, and one or more folds between the first end portion 612 and the second end portion 614 flexible linker 106.

A force of approximately 0.1 Newton applied to the first end portion 612 of the flexible linker 106 may cause the second end portion 614 thereof to move by approximately 5-75 degree.

The length of the flexible linker 106 may be approximately between 1 cm and 1 m.

The flexible linker 106 may be made of nitinol, stainless steel, titanium, any shape-memory material, any biocompatible alloys, any biocompatible polymers, and the like, as well as any combination(s) thereof.

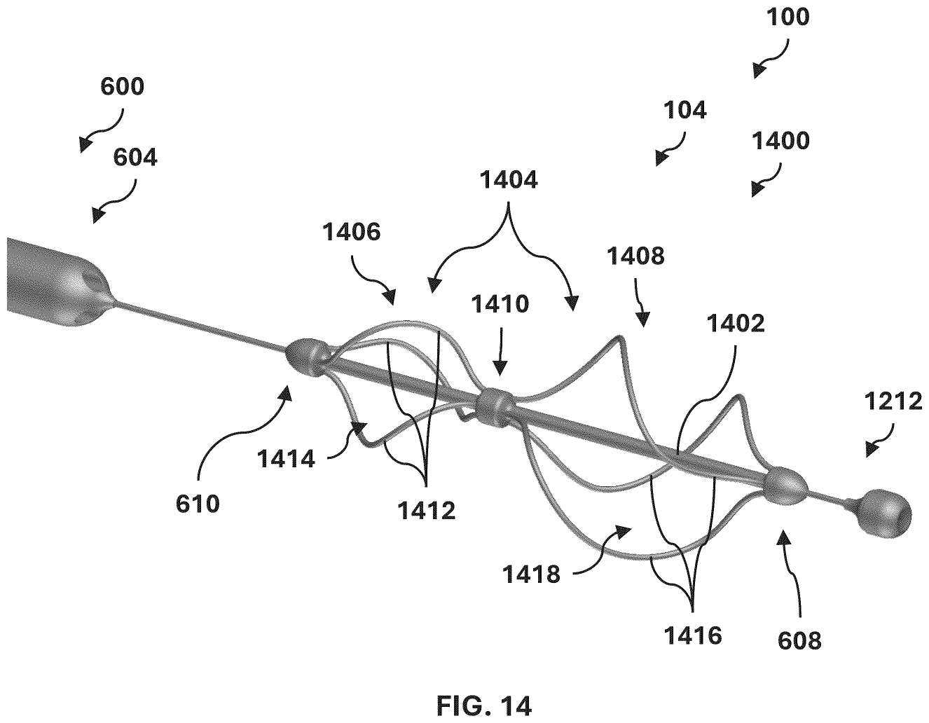

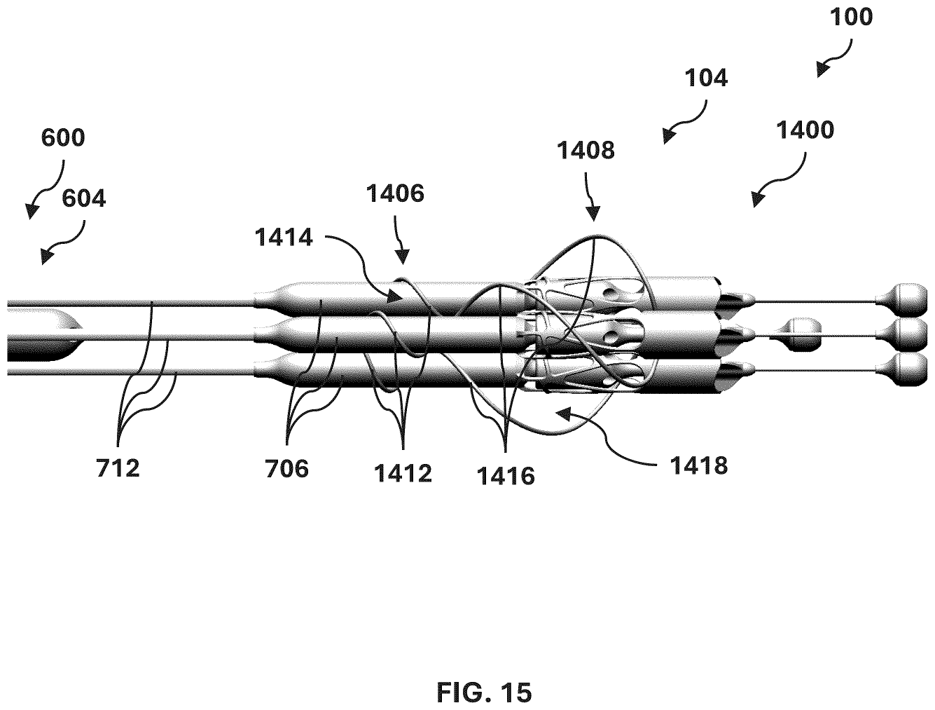

FIGS. 14-15 illustrate the second medical device component 104 optionally provided as another anchorable medical device, such as an anchor 1400, according to one or more embodiment(s). In particular, FIG. 14 illustrates the anchor 1400 without any assemblable medical device, and FIG. 15 illustrates the anchor 1400 with three assemblable medical devices assembled to the anchor 1400.

The anchor 1400 includes a post 1402 having the first and second end portions 608, 610, and a helical-like structure 1404 at least partially extending between the first and second end portions 608, 610 of the anchor 1400. The helical-like structure 1404 includes a first helical-like portion 1406 and a second helical-like portion 1408. The first helical-like portion 1406 includes three strands that are directly or indirectly coupled to and extends between the second end portion 610 and an intermediate portion 1410 of the post 1402. The second helical-like portion 1408 also includes three strands that are directly or indirectly coupled to and extends between the intermediate portion 1410 and the first end portion 608 of the post 1402. The strands of the first helical-like portion 1406 are radially arranged closer to the post 1402 as compared to the strands of the second helical-like portion 1408. Alternatively, each of the first helical-like portion 1406 and the second helical-like portion 1408 may include any number of strands so arranged.

The anchor 1400 and the anchor 606 being generally similar to each other, the anchor 1400 will be generally described herein. It will appreciate that the description of the anchor 606 applies to the anchor 1400 with the necessary change(s), appreciable to the skilled addressee, having been made, if applicable.

Still referring to FIGS. 14-15, each strand of the first helical-like portion 1406 forms an assembling loop 1412 (also referred to herein as an “assembling element”) configured to intracorporeally assemble a respective assemblable pump 700 in a subject's body, according to one or more embodiment(s). In particular, each assembling loop 1412 (also referred to herein as an “assembling element”; only one opening 1414 is labelled in each of FIGS. 14-15 for the sake of clarity) forms a respective opening 1414 configured to slidably receive a respective electrical lead 712 therethrough and further configured to slidably receive and/or slidably engage a respective pump unit 706 therein.

As illustrated in FIG. 15, each pump unit 706 of three assemblable pumps 700 are received or engaged in a respective opening 1414, according to one or more embodiment(s). Slidably received in and/or slidably engaged with the opening 1414, each pump unit 706 may be free-floating or may be immobilized. Alternatively, each strand of the first helical-like portion 1406 may form multiple assembling loops and corresponding openings configured to intracorporeally assemble to one or more respective assemblable pumps 700.

As illustrated in FIGS. 14-15, each strand of the second helical-like portion 1408 forms an anchoring loop 1416 (also referred to herein as an “anchoring element) configured to intracorporeally anchor the anchor 1400 in a subject's body, according to one or more embodiment(s). Each anchoring loop 1416 forms a respective opening 1418 configured to slidably receive a respective electrical lead 712 therethrough and a respective pump unit 706 therein (only one opening 1418 is labelled in each of FIGS. 14-15 for the sake of clarity). In particular, as illustrated in FIG. 15, each pump unit 706 of the three assemblable pumps 700 is slidably received in a respective opening 1418, according to one or more embodiment(s). So slidably received, each functional unit 112 is free-floating.

Generally similarly to the anchoring loops 1202, the anchoring loops 1416 may or may not be configured to exert pressure to the lumen wall for engaging the lumen wall in a way that the anchor 1400 may be moved therealong without traumatizing or damaging the lumen wall.

Alternatively, each strand of the second helical-like portion 1408 may form multiple anchoring loops 1416 and corresponding openings 1418 configured to slidably receive a respective electrical lead 712 therethrough and a respective pump unit 706 therein.

Still alternatively, the strands of the first helical-like portion 1406 may be radially arranged farther from the post 1402 as compared to the strands of the second helical-like portion 1408, such that the first helical-like portion 1406 has the anchoring loops 1416, and the second helical-like portion 1408 has the assembling loops 1412. Also, another second helical-like portion 1408 may be provided to the second end portion 610 of the post 1402, such that the first helical-like portion 1406 is positioned between two second helical-like portions 1408. In the last case, generally similarly to the first and second clusters 1206, 1208 of anchoring loops 1202, two second helical-like portions 1408 better maintain the post 1402 in a coaxial alignment with the lumen, as opposed to a single second helical-like portion 1408 of anchoring loops 1416.

Also generally similarly to the assembling loops 1204 and the anchoring loops 1202, the assembling loops 1412 and the anchoring loops 1416 have an expanded configuration (also referred to herein as a “deployed configuration”), as illustrated in FIGS. 14-15, and a non-expanded configuration (also referred to herein as an “undeployed configuration”), according to one or more embodiment(s). Moreover, as illustrated in FIG. 15, the pump units 706 assembled to the anchor 1400 in the expanded configuration are partially surrounded by or enclosed in the anchoring loops 1416, according to one or more embodiment(s). Alternatively, the pump units 706 assembled to the anchor 1600 in the expanded configuration may be entirely surrounded by or enclosed in the anchoring loops 1416.

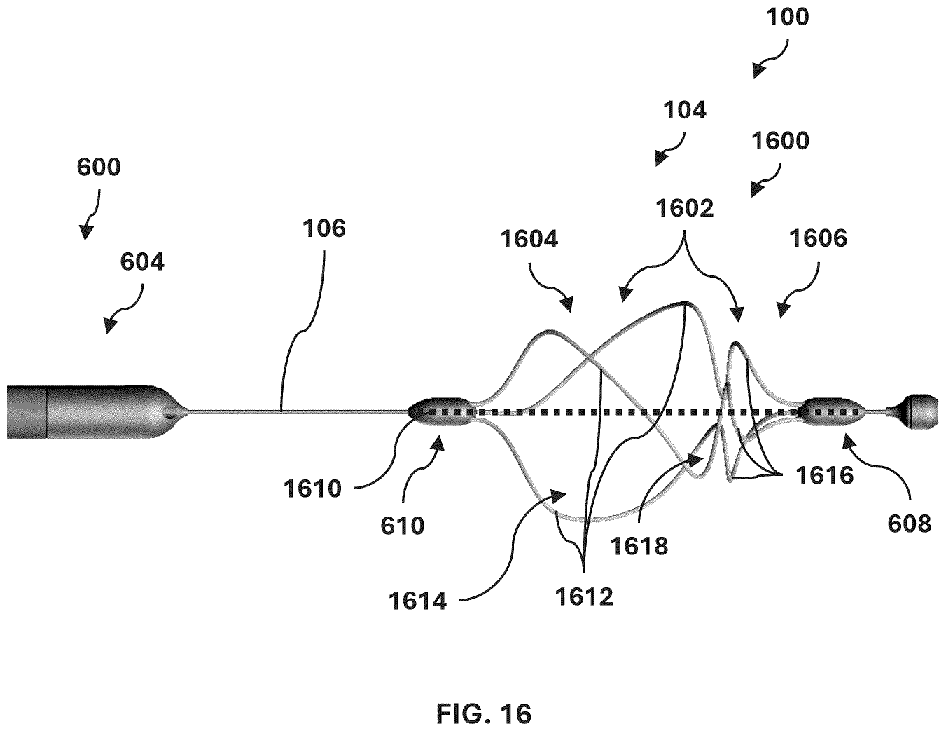

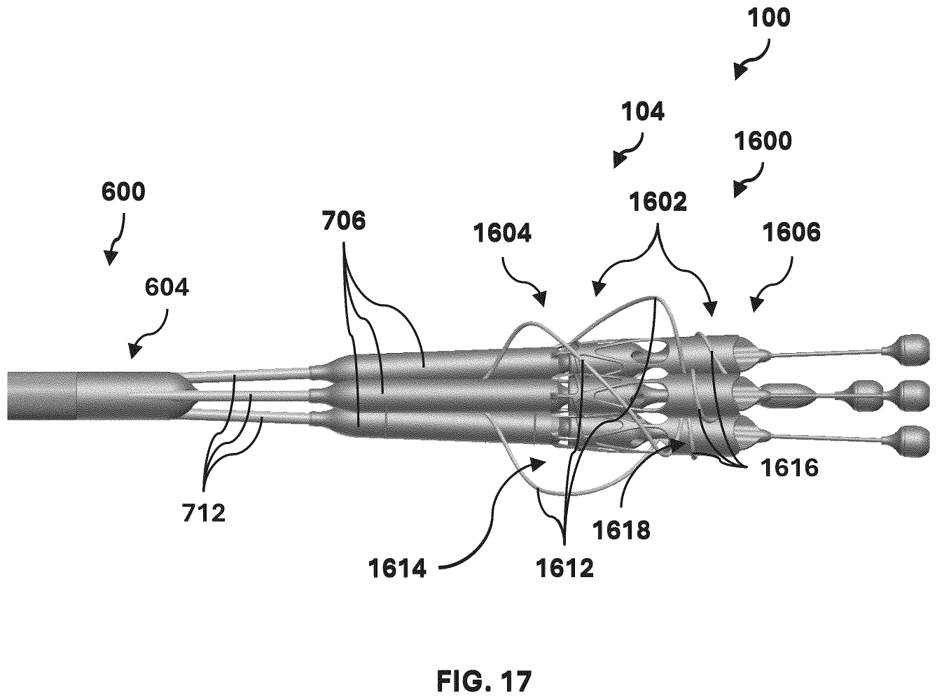

FIGS. 16-17 illustrate the second medical device component 104 optionally provided as another anchorable medical device, such as an anchor 1600, according to one or more embodiment(s). In particular, FIG. 16 illustrates the anchor 1600 without any assemblable medical device, and FIG. 17 illustrates the anchor 1600 with three assemblable medical devices assembled to the anchor 1600.

The anchor 1600 includes a helical-like structure 1602 provided with three strands forming a first helical-like portion 1604 and a second helical-like portion 1606, and extending between the first end portion 608 and the second end portion 610 of the anchor 1600. The portions of the strands that forms the first helical-like portion 1604 are radially arranged farther from a central reference axis 1610 (represented in FIG. 16 by a dashed line) as compared to the strands that forms the second helical-like portion 1606. Alternatively, each of the first helical-like portion 1604 and the second helical-like portion 1606 may include any number of strands so arranged.

The anchor 1600 as well as the anchors 606 and 1400 being generally similar to each other, the anchor 1600 will be generally described herein. It will appreciate that the descriptions of the anchors 606 and 1400 apply to the anchor 1600 with the necessary change(s), appreciable to the skilled addressee, having been made, if applicable.

Still referring to FIGS. 16-17 each strand of the first helical-like portion 1604 forms an anchoring loop 1612 (also referred to herein as an “anchoring element”) configured to intracorporeally anchor the anchor 1600 in a subject's body, according to one or more embodiment(s). Each anchoring loop 1612 forms a respective opening 1614 (only one opening 1614 is labelled in each of FIGS. 16-17 for the sake of clarity) configured to slidably receive a respective electrical lead 712 therethrough and optionally a pump unit 706 of the three assemblable pumps 700 is slidably received in a respective opening 1614, according to one or more embodiment(s). So slidably received, each functional unit 112 may be free-floating.

Generally similarly to the anchoring loops 1202, the anchoring loops 1612 may or may not be configured to exert pressure to the lumen wall for engaging the lumen wall in a way that the anchor 1600 may be moved therealong without traumatizing or damaging the lumen wall.

Alternatively, each strand of the first helical-like portion 1604 may form multiple anchoring loops 1612 and corresponding openings 1614 configured to slidably receive a respective electrical lead 712 therethrough and a respective pump unit 706 therein.

As illustrated in FIGS. 16-18, each strand of the second helical-like portion 1606 forms an assembling loop 1616 (also referred to herein as an “assembling element”) configured to intracorporeally assemble a respective assemblable pump 700 in a subject's body, according to one or more embodiment(s). In particular, each assembling loop 1616 (also referred to herein as an “assembling element”; only one opening 1618 is labelled in each of FIGS. 16-17 for the sake of clarity) forms a respective opening 1618 configured to slidably receive a respective electrical lead 712 therethrough and further configured to slidably receive and/or slidably engage a respective pump unit 706 therein.

As illustrated in FIG. 17, each pump unit 706 of three assemblable pumps 700 are received or engaged in a respective opening 1618, according to one or more embodiment(s). Slidably received in and/or slidably engaged with the opening 1618, each pump unit 706 may be free-floating or may be immobilized. Alternatively, each strand of the second helical-like portion 1606 may form multiple assembling loops 1616 and corresponding openings 1618 configured to intracorporeally assemble to one or more respective assemblable pumps 700.

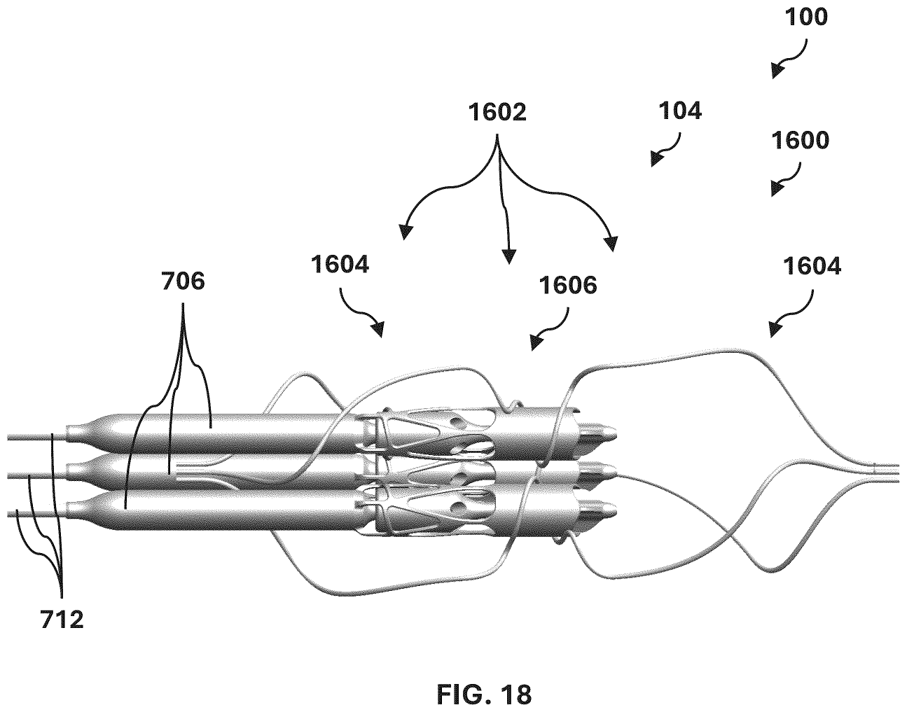

Still alternatively, the strands of the second helical-like portion 1606 may be radially arranged farther from the central reference axis 1610 as compared to the strands of the first helical-like portion 1604, such that the second helical-like portion 1606 has the assembling loops 1616, and the first helical-like portion 1604 has the anchoring loops 1612. Also, as illustrated in FIG. 18, another first helical-like portion 1604 may be provided to the first end portion 608 of the anchor 1600, such that the second helical-like portion 1606 is positioned between two first helical-like portions 1604. In the last case, generally similarly to the first and second clusters 1206, 1208 of the anchoring loops 1202, two first helical-like portions 1604 better maintain the anchor 1600 in a coaxial alignment with the lumen, as opposed to a single first helical-like portion 1406 of anchoring loops 1612. It is to be noted that the catheter 600 and capturable element 714 are omitted from FIG. 18.

Also, generally similarly to the assembling loops 1204 and the anchoring loops 1202, the assembling loops 1616 and the anchoring loops 1612 have an expanded configuration (also referred to herein as a “deployed configuration”), as illustrated in FIGS. 16-17, and a non-expanded configuration (also referred to herein as an “undeployed configuration”), according to one or more embodiment(s). Moreover, as illustrated in FIG. 17, the assemblable pumps 700 assembled to the anchor 1600 in the expanded configuration are partially surrounded by or enclosed in the anchoring loops 1612, according to one or more embodiment(s). Alternatively, the assemblable pumps 700 assembled to the anchor 606 in the expanded configuration may be entirely surrounded by or enclosed in the anchoring loops 1202.

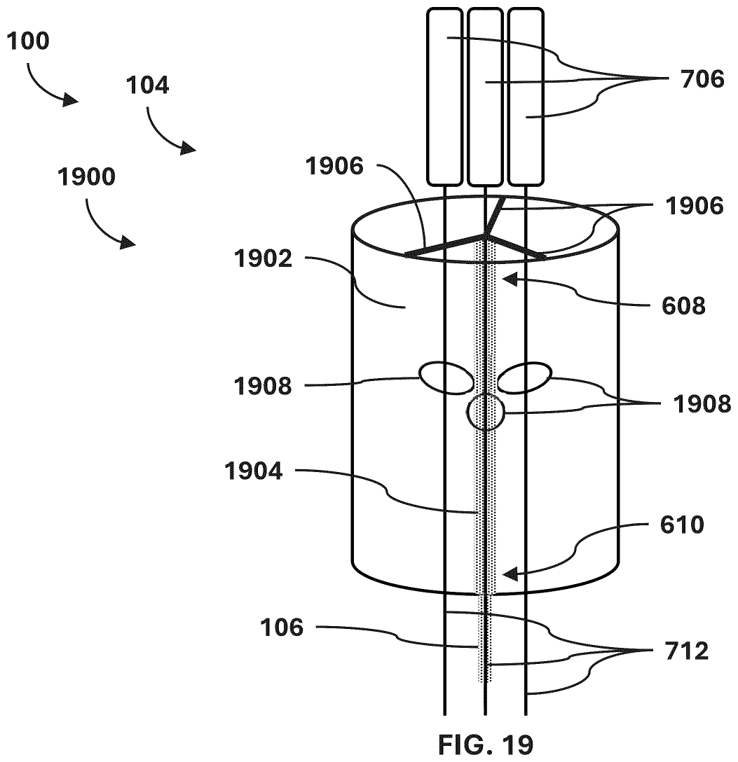

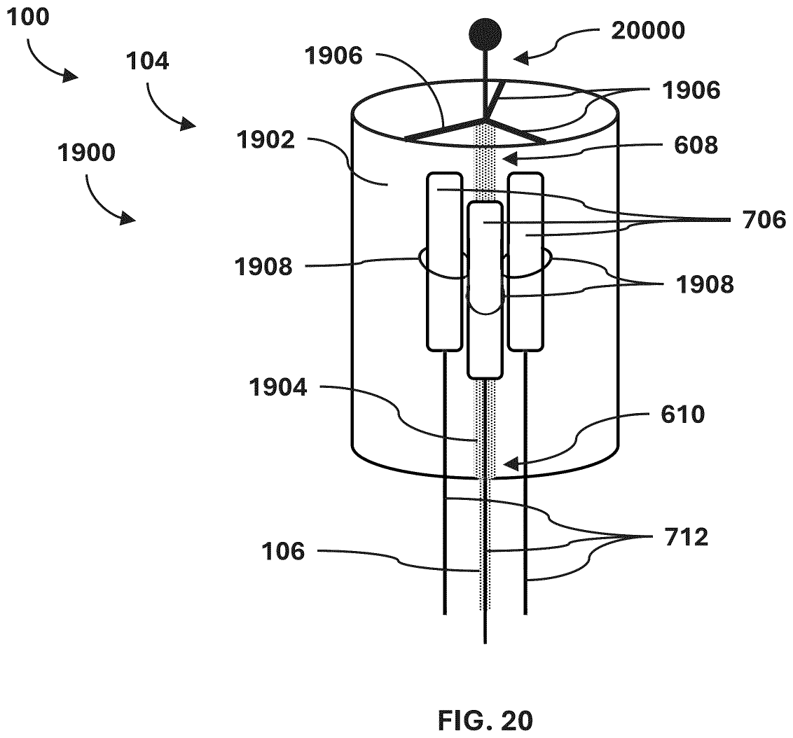

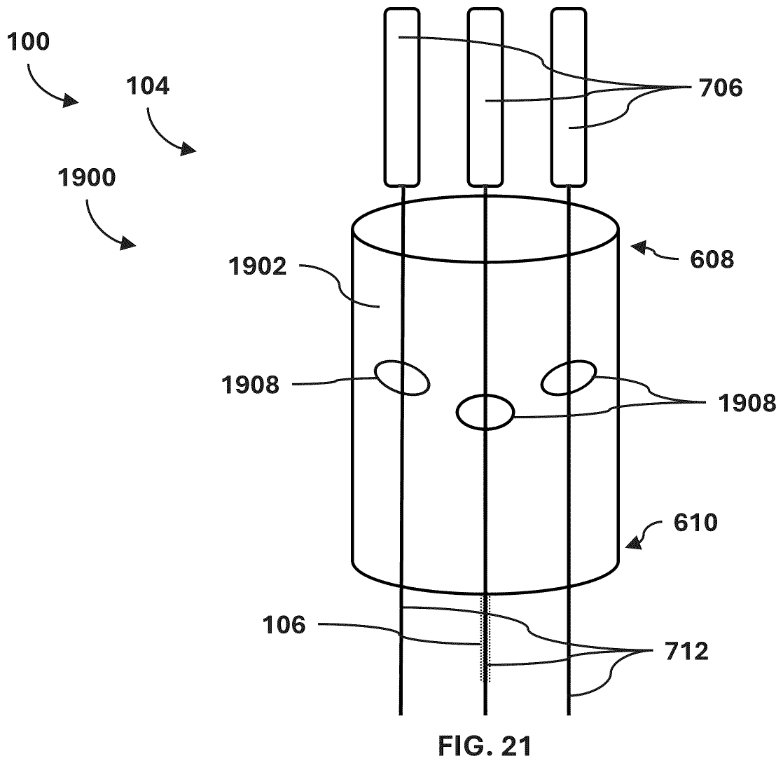

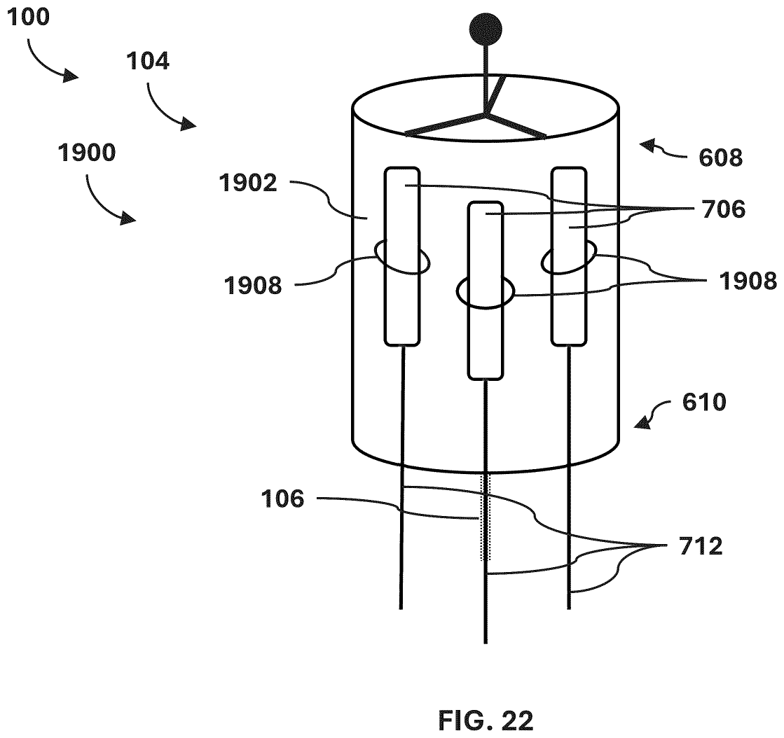

FIGS. 19-22 illustrate the second medical device component 104 optionally provided as another anchorable medical device, such as an anchor 1900, according to one or more embodiment(s). In particular, FIGS. 19-20 illustrate the anchor 1900 provided with a stent-like structure 1902 and an optional post 1904 positioned within the stent-like structure 1902. FIGS. 21-22 illustrate the anchor 1900 with the stent-like structure 1902 alone, without any post. FIGS. 19 and 21 illustrate the anchor 1900 unassembled from three assemblable pumps 900. FIGS. 20 and 22 illustrate the anchor 1900 assembled to three assemblable pumps 900. The stent-like structure 1902 has a tubular wall having an outer wall surface configured to engage an anatomical structure, such as a lumen wall. The tubular wall of the stent-like structure 1902 is illustrated as transparent in FIGS. 19-22 as a way to see through it.

The anchor 1900 and the anchor 606 being generally similar to each other, the anchor 1900 will be generally described herein. It will appreciate that the description of the anchor 606 applies to the anchor 1900 with the necessary change(s), appreciable to the skilled addressee, having been made, if applicable.

As illustrated in FIGS. 19-20, the post 1904 extends through a longitudinal bore (also referred to herein as an “opening”) of the stent-like structure 1902 between the first end portion 608 and the second end portion 610 of the anchor 1900, is attached at the first end portion 608 by three arms 1906 to the stent-like structure 1902, and is further attached at the second end portion 610 to the flexible linker 106, according to one or more embodiment(s). Alternatively, any number of arms may be provided anywhere along the post 1904 and may attach anywhere to the stent-like structure 1902. The anchor 1900 includes three assembling loops 1908 (also referred to herein as an “assembling element”) provided to the post 1904. Each assembling loop 1908 may be directly or indirectly attached to the post 1904.

As illustrated in FIGS. 21-22, the anchor 1900 has no post, and the flexible linker 106 is attached to the second end portion 610 of the stent-like structure 1902 by any number of arms 1906 (not shown), according to one or more embodiment(s). The anchor 1900 includes three assembling loops 1908 (also referred to herein as an “assembling element”) provided to an inner wall surface of the tubular wall. Each assembling loop 1908 may be directly or indirectly coupled to the tubular wall.

Referring to FIGS. 19-22, the tubular wall of the stent-like structure 1902 may or may not be configured to exert pressure to a lumen wall for the outer wall surface (also referred to herein as an “anchoring element” having an “engagement portion”) of the tubular wall to engage the lumen wall in a way that the anchor 1900 may be moved therealong without traumatizing or damaging the lumen wall, as described herein.

Further, as illustrated, the longitudinal bore of the stent-like structure 1902 is configured to slidably receive the electrical leads 712 of the assemblable pumps 900 therethrough, with each electrical lead 712 passing through a respective passage defined by two adjacent arms 1906, as the case may be. The three assembling loops 1908 are configured to intracorporeally assemble and unassemble a respective pump unit 706 in a subject's body, as described herein. The assembling and unassembling of the pump units 706 are as described herein.

Any number of assemblable medical devices 110, such as the assemblable pumps 900, may be provided to the implantable medical device 100. Depending on the arrangement of the second medical device component 104, such as the anchors 606, 1400, 1600, 1900, any number of assembling elements, such as the assembling loops 1204, 1412, 1616, 1908, may be provided to the second medical device component 104 for assembling any number of assemblable medical devices 110. Any number of assembling loops 1908 may be provided to each arm 1906 of the anchor 1900. In particular, a single assemblable medical device 110 and one or more assembling element may be configured to assemble together and unassemble for each other. A single assembling element and one or more assemblable medical device 110 may be configured to assemble together and unassemble for each other.

The stent-like structure 1902, the arms 1906, and the assembling loops 1908 have an expanded configuration, as illustrated in FIGS. 19-22, and a non-expanded configuration, according to one or more embodiment(s).

In the expanded configuration, the anchor 1900 is configured to intracorporeally anchor itself and to intracorporeally assemble to the pump units 706, such as in the lumen of a subject's body conduit. The stent-like structure 1902 has its tubular wall deployed and is configured to engage a lumen wall. The arms 1906 and the assembling loops 1908 generally radially project from the post 1904 and/or from the inner wall surface of the tubular wall, as the case may be (also referred to herein as a “deployed configuration”). The assembling loops 1908 are configured to assemble to corresponding functional units 706 of the assemblable medical devices 110.

In the non-expanded configuration, the anchor 1900 is configured for intracorporeal delivery of the implantable medical device 100. For example, one or more assemblable medical devices may be intracorporeally sheathable in a subject's body, for example for retrieving or explanting the implantable medical device or an assemblable medical device. The stent-like structure 1902 is generally collapsed and may have its tubular wall positioned closer to the post 1904 compared to when the tubular wall is deployed. The arms 1906 and the assembling loops 1908 are generally positioned along the inner surface of the tubular wall and the post 1904, as it may be the case (also referred to herein as an “undeployed configuration”). So positioned, the arms 1906 and/or the assembling loops 1908 may project toward the first end portion 608 of the anchor 606 outside the longitudinal bore of the tubular wall, or toward the second end portion 610 of the anchor 606 inside the longitudinal bore of the tubular wall.

When made of a shape-memory material and overcomeably biased toward the expanded configuration, the anchor 1900 may be converted from the expanded configuration to the non-expanded configuration by introducing the anchor 1900 in a sheath, for example. In this case, the first end portion 608 of the anchor 1900 may have a chamfer or be rounded to facilitate the introduction. As such, the anchor 1900 is self-expandable. When made of a non-shape-memory material, the anchoring loops 1908 may be converted from the non-expanded configuration to the expanded configuration by inflating a balloon catheter appropriately positioned in the longitudinal bore of the tubular wall, for example. The anchor 1900 may be converted between the non-expanded configuration and the expanded configuration, and vice versa, in a lumen of a subject's body conduit, such as in the vascular system.

As illustrated in FIGS. 20 and 22, the assemblable pumps 700 assembled to the anchor 1900 in the expanded configuration are surrounded by the stent-like structure 1902, according to one or more embodiment(s). Such arrangement generally prevents or mitigates any undesired contact of the assemblable pumps 700 with the lumen wall, as described herein. Alternatively, the assemblable pumps 700 assembled to the anchor 1900 in the expanded configuration may be partially surrounded by the stent-like structure 1902, such that one or more assemblable pumps 700 project outside the longitudinal bore of the tubular wall at the first end portion 608 and/or at the second end portion 610 of the anchor 1900. Depending on the assemblable pumps 700, this last arrangement may also generally prevents or mitigates any undesired contact of the assemblable pumps 700 with the lumen wall.

As illustrated in FIGS. 20 and 22, the anchor 1900 optionally includes a capturable element 20000 attached to the first end portion 608 of the anchor 1900 by three arms 1906, and configured to be intracorporeally captured by a medical tool, as described herein, according to one or more embodiment(s). The capturable element 20000 is omitted from FIGS. 21 and 22 for the sake of clarity.

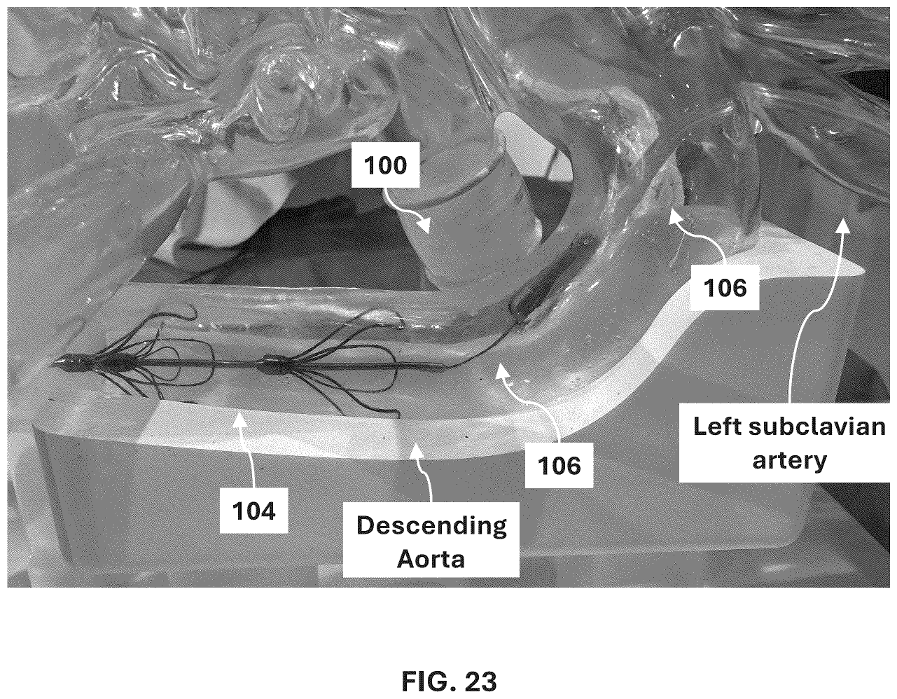

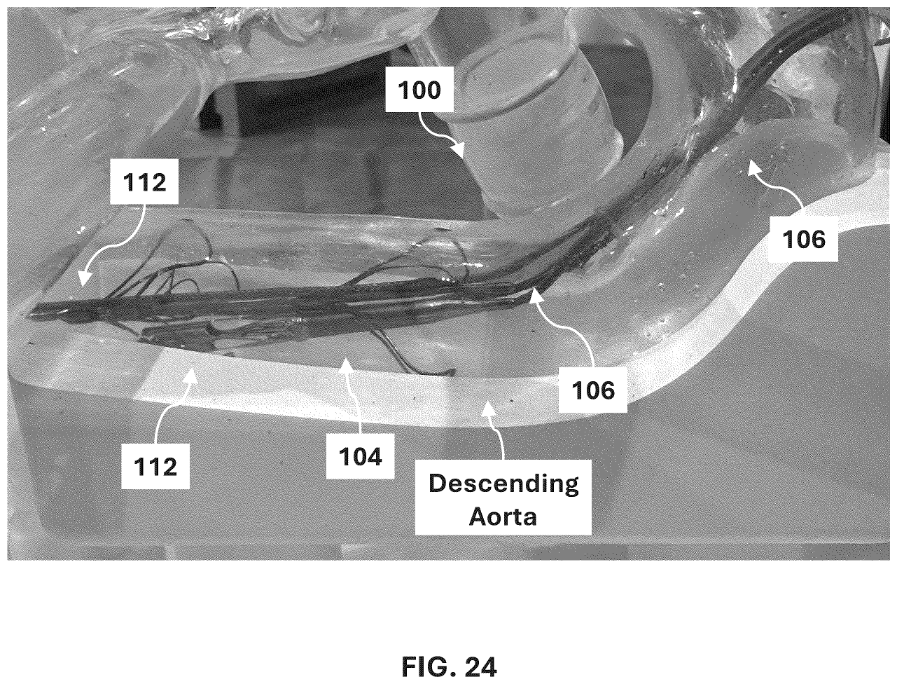

In complement to the schematical representations of FIGS. 2-5, FIGS. 23-24 present the implantable medical device 100 provided with the catheter 600 and the anchor 606, and implanted in a portion of a mock anatomical vascular system, according to an aspect of this disclosure. In particular, FIGS. 23 and 24 present the anchor 606 without any assemblable pump and with the pump units 706 of two assemblable pumps 700 assembled thereto, respectively, according to one or more embodiment(s). Only two instead of three assemblable pumps 700 are represented in FIG. 24 for the sake of clarity.

The implantable medical device 100 is implanted such that the anchor 606 and the two assemblable pumps 700 are positioned inside a descending aorta, while the catheter 600 is routed from the anchor 606 up to a left subclavian artery and ultimately outside the mock anatomical vascular system. As illustrated in FIGS. 23-24, the flexible linker 106 and the two electrical leads 712 of the two assemblable pumps 700 are flexed or bended to appropriately accommodate an angle defined by the descending aorta. Absent the flexible linker 106 and with the anchor 606 attached to the catheter 600, the catheter 600 could not pass through the angle for the anchor 606 to be appropriately positioned and/or anchored inside the descending aorta. The two pump units 706 are also positioned for appropriate operation inside the descending aorta. It is to be noted that the left subclavian artery of the mock anatomical vascular system is only visible in FIG. 23.

The implantable medical device 100 may be intracorporeally implanted in a subject's body in a manner similar to its implantation in the mock anatomical vascular system of FIGS. 23-24, according to one or more embodiment(s). In this case, the second end portion 604 of the catheter 600 is externalized outside the subject's body through a subject's body intracorporeal access, such as an axillary or subclavian intracorporeal access. So externalized, each electrical lead 712, which are slidably received through the catheter 600, may be manipulated at the second end portion 604 of the catheter 600 for assembling and unassembling a respective pump unit 706 to the anchor 606 inside the aorta. Further, the second end portion 710 of each electrical lead 712 may be connected to a controller (not shown) for operating a respective pump unit 706 inside the aorta, for example to provide a hemodynamic effect.

The implantable medical device 100 may be a transcatheterly implantable medical device.

The implantable medical device 100 may include any one of the medical devices listed under LIST 1 hereinafter that is provided with the flexible linker 106 between two or more of its components.

The implantable medical device 100 may also include any one of the medical devices listed under LIST 1 hereinafter which are provided as the first medical device component 102 and/or as the second medical device component 104.

The implantable medical device 100 may include one or more assemblable medical devices, and the assemblable medical device(s) may include any one of the medical devices listed under LIST 1 hereinafter.

LIST 1: a blood pump; a percutaneous ventricular assist device; a stent; a stent-graft; a catheter or cannula; an intra-aortic balloon, including an intra-aortic balloon counter pulsation; an endovascular or stent graft; a vascular closure device; an embolic protection device; an occluder, including a left atrial appendage occluder; a drug-eluting device; a pacemaker; an implantable cardioverter defibrillator; an insertable or implantable monitoring device, such as an insertable or implantable hemodynamic monitoring device; a valve, such as a heart valve, and a related delivery system; a valvuloplasty device; an annuloplasty device; an annuloplasty ring; and/or a prosthetic valve chords.

According to another aspect, this disclosure generally relates solely to an anchor that is similar to any one of the anchors 606, 1400, 1600, 1900 described herein, provided or not with the flexible linker 106. In this case, such anchor may also be referred to herein as an “anchorable and assemblable medical device, structure, or unit” or as an “assemblable medical device anchor”. Such anchor may or may not be provided with any number of assemblable medical devices, according to one or more embodiment(s).

Still according to another aspect, this disclosure generally relates to an implantable medical device that is similar to the implantable medical device 100 described herein, but provided without the flexible linker 106 and with the first medical device component 102 directly or indirectly coupled to the second medical device component 104. Such implantable medical device may or may not be provided with any number of assemblable medical devices, according to one or more embodiment(s).

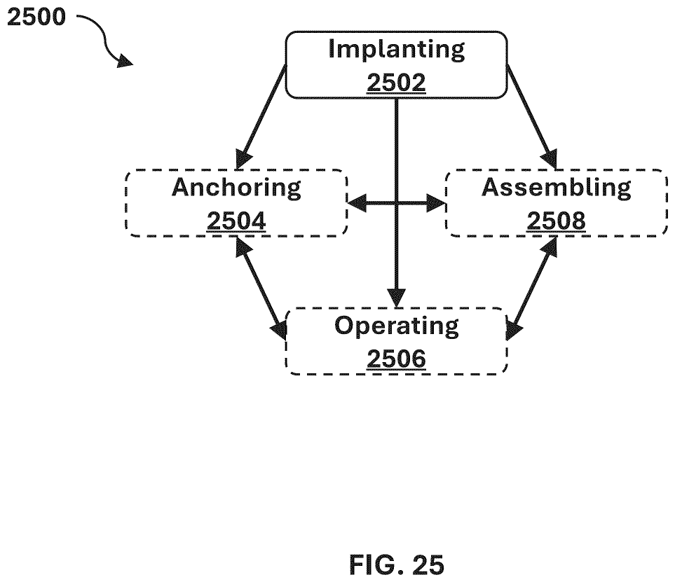

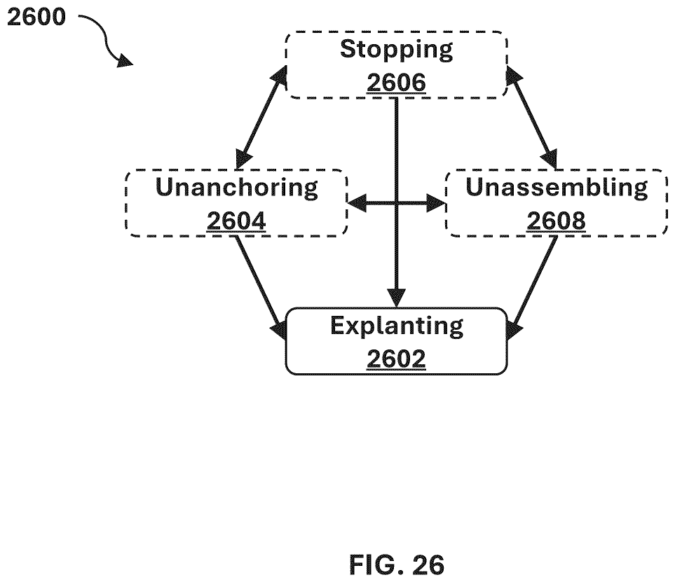

Turning now to FIGS. 25-26, methods of using an implantable medical device will be described, according to an aspect of this disclosure. In particular, FIG. 25 generally relates to a method of implanting an implantable medical device, and FIG. 26 generally relates to a method of explanting an implanted medical device, according to one or more embodiment(s). Optional feature(s) is/are represented by corresponding dashed line box(es) in FIGS. 25-26. It is to be noted that each and every reference to the implantable medical device 100 and other feature(s), as the case may be, is solely made to facilitate an understanding of how these methods may be implemented and practiced. Accordingly, such reference is not intended to limit the scope of such methods in any way.

According to the methods 2500 and 2600, an end portion of the linker (e.g., the flexible linker 106) is directly or indirectly coupled to a first medical device component (e.g., the first medical device component 102) of the implantable medical device (e.g., the implantable medical device 100), and another end portion of the linker is be directly or indirectly coupled to a second medical device component (e.g., the second medical device component 104) of the implantable medical device.

Further, the methods 2500 and 2600 may rely on a first subject's body access and/or on a second subject's body access, as the case may be, for implantation and explantation, respectively. Each of the first subject's body access and the second subject's body access may be a subject lower body access or a subject upper body access. Each of the first subject's body access and the second subject's body access may be a subject natural body access or a subject surgically made body access. For example, the first subject's body access may be a subject lower body access, such as a subject femoral access, and the second subject's body access may be subject upper body access, such as an axillary or subclavian access.

As schematically represented in FIG. 25, the method 2500 of implanting an implantable medical device (e.g., the implantable medical device 100; also referred to herein as a “medical device”) in a subject's body includes: implanting 2502 the implantable medical device in the subject's body to cause a linker thereof (e.g., the flexible linker 106) to transition between a first configuration and a second configuration, according to one or more embodiment(s).

For example, the first configuration may be a generally linear conformation or arrangement, and the second configuration may be a generally nonlinear conformation or arrangement. Conversely, the first configuration may be a generally nonlinear conformation or arrangement, and the second configuration may be a generally linear conformation or arrangement.

The first configuration may be a generally curved conformation or arrangement, and the second configuration may be a generally non-curved conformation or arrangement. Conversely, the first configuration may be a generally non-curved conformation or arrangement, and the second configuration may be a generally curved conformation or arrangement.

The first configuration may be a generally angled conformation or arrangement, and the second configuration may be a generally non-angled conformation or arrangement. Conversely, the first configuration may be a generally non-angled conformation or arrangement, and the second configuration may be a generally angled conformation or arrangement.

The first configuration may be a less resilient conformation or arrangement, and the second configuration may be a comparatively more resilient conformation or arrangement. Conversely, the first configuration may be a more resilient conformation or arrangement, and the second configuration may be a comparatively less resilient conformation or arrangement. This may be the case when the resilience property of the linker varies as the linker is transitioned between the first configuration and the second configuration, such that the linker has different resilience properties or capacities in the first configuration compared to the second configuration.

Referring back to FIGS. 2-3, for example, implanting 2502, may include implanting the implantable medical device in a subject vascular system, such that the first medical device component is at least partially located in the left subclavian artery and/or the aortic arch, while the second medical device component is at least partially located in in the descending aorta, as illustrated in FIGS. 2-3, but also in the ascending aorta and/or the aortic arch., according to one or more embodiment(s).

Referring back to FIGS. 4-5, for example, implanting 2502 may include implanting the implantable medical device in a subject vascular system, such that the linker is received through the orifice of the aortic valve, while the first medical device component is located in the ascending aorta, as illustrated in FIGS. 4-5, but also in the aortic arch and/or the descending aorta, and the second medical device component is located in the left ventricle, according to one or more embodiment(s).

Implanting 2502 may include implanting the implantable medical device in a subject vascular system according to the following modalities:
(i) the linker is at least partially located in the left common carotid artery and/or the aorta, while the first medical device component is at least partially located in the left common carotid artery, and the second medical device component is at least partially located in the aorta;
(ii) the linker is at least partially located in the brachiocephalic trunk and/or the aorta, while the first medical device component is at least partially located in the brachiocephalic trunk, and the second medical device component is at least partially located in the aorta;
(iii) the linker is at least partially located in a radial artery and/or the aorta, while the first medical device component is at least partially located in the radial artery, and the second medical device component is at least partially located in the aorta;
(iv) the linker is at least partially located in the aorta and/or one of the renal arteries, while the first medical device component is at least partially located in the aorta, and the second medical device component is at least partially located in a corresponding one of the renal arteries;
(v) the linker is at least partially located in the inferior vena cava and/or one of the renal veins, while the first medical device component is at least partially located in the inferior vena cava, and the second medical device component is at least partially located in a corresponding one of the renal veins;
(vi) the linker is at least partially located in the aorta and/or one of the femoral arteries, while the first medical device component is at least partially located in the aorta, and the second medical device component is at least partially located in the femoral artery;
(vii) the linker is at least partially located in the inferior vena cava and/or one of the femoral veins, while the first medical device component is at least partially located in the inferior vena cava, and the second medical device component is at least partially located in the femoral veins;
(viii) the linker is passed from the inferior vena cava to the aorta, while the first medical device component is at least partially located in the inferior vena cava, and the second medical device component is at least partially located in the aorta;
(ix) the linker is at least partially located in one of the jugular veins, the left or right brachiocephalic vein, the superior vena cava, and/or the right ventricle, while the first medical device component is at least partially located in a corresponding one of the jugular veins, and the second medical device component is at least partially located the right ventricle;
(x) the linker is at least partially located in one of the jugular veins, the left or right brachiocephalic vein, the superior vena cava, the right ventricle, and/or through the orifice(s) of at least one of the tricuspid valve and the pulmonary valve, while the first medical device component is at least partially located in a corresponding one of the jugular veins, and the second medical device component is at least partially located in the left or right pulmonary artery;
(xi) the linker is at least partially located in the left or right brachiocephalic vein, the superior vena cava, and/or the right atrium, while the first medical device component is at least partially located in a corresponding one of the left and right brachiocephalic veins, and the second medical device component is at least partially located in the right atrium;
(xii) the linker is at least partially located in the left or right brachiocephalic vein, the superior vena cava, the right atrium, through the orifice of the tricuspid valve, and/or the right ventricle, while the first medical device component is at least partially located in a corresponding one of the left and right brachiocephalic veins, and the second medical device component is at least partially located in the right ventricle;
(xiii) the linker is received through the orifice of the mitral/bicuspid valve, while the first medical device component is at least partially located in the left ventricle, and the second medical device component is at least partially located in the left atrium;
(xiv) the linker is received through the orifice of the aortic valve and the mitral/bicuspid valve, while the first medical device component is at least partially located in the aorta, and the second medical device component is at least partially located in the left atrium;
(xv) the linker is received through the orifices of the aortic valve, the mitral/bicuspid valve, and the atrial septum, while the first medical device component is at least partially located in the aorta, and the second medical device component is at least partially located in the inferior or superior vena cava; and
(xvi) the linker is disposed through the atrial septum, while the first medical device component is at least partially located in the left atrium, and the second medical device component is at least partially located in the right atrium.

Implanting 2502 may cause the linker to momentarily transition from the first configuration to the second configuration, and vice-versa. For example, this may be the case when the implantable medical device is introduced, routed, and/or navigated in a lumen of a subject's body conduit, causing the linker to momentarily flex, bend, articulate, pivot, hinge, or swivel for the implantable medical device to accommodate a tortuous portion of the lumen along the way.

Implanting 2502 may also cause the linker to remain transitioned from the first configuration to the second configuration, and vice-versa, for the duration of its use. For example, this may be the case when the implantable medical device is delivered, anchored, assembled, operated, and/or implanted in a lumen of a subject's body conduit, causing the linker to remain so transitioned until explantation or repositioning.

Implanting 2502 may include transcatheterly implanting the implantable medical device in the vascular system of the subject's body. In particular, an optional guidewire of the implantable medical device is introduced through a first subject's body access in the vascular system and routed therein up to an intraluminal location. The optional guidewire is captured or snared at the intraluminal location by a snare having been introduced through a second subject's body access in the vascular system and having been routed therein up to the optional guidewire.

Then, the implantable medical device is implanted by navigating the implantable medical device up to an intraluminal implantation site. In particular, the implantable medical device is introduced, such as by pushing it, through the first subject's body access in the vascular system, and/or by pulling the snare from the second subject's body access, to navigate the implantable medical device adjacent or up to the intraluminal implantation site. In doing so, a portion of the implantable medical device may or may not be at least partially externalized through the second subject's body access. The snare may be detached from the implantable medical device inside the subject's body, when the implantable medical device remains in the subject's body, or outside the subject's body, when the implantable medical device is externalized.

For example, the guidewire may be provided to the first medical device component 102 of the implantable medical device (e.g., to the second end portion 604 of the catheter 600). An atraumatic end portion (e.g., the second end portion 604 of the catheter 600) of the first medical device component 102 may be introduced through the first subject's body access. The first medical device component 102 may be introduced through the first subject's body access before the second medical device component 104 is introduced through the first subject's body access.

Alternatively, in absence of the optional guidewire, the implantable medical device is introduced, such as by pushing it, through the first subject's body access in the vascular system and navigated therein up to the intraluminal implantation site. As described herein, doing so, a portion of the implantable medical device may or may not be at least partially externalized through the second subject's body access, and the snare may be detached from the implantable medical device inside or outside the subject's body.

For example, the first medical device component 102 or the second medical device component 104 of the implantable medical device, which may be provided with an atraumatic portion, may be introduced through the first subject's body access in the vascular system. The first medical device component 102 may be introduced through the first subject's body access before the second medical device component 104 is introduced through the first subject's body access.

Either provided with an optional guidewire or not, the implantable medical device may alternatively be at least partially enclosed in a delivery sheath (also referred to herein as “sheathed”) for implantation. In particular, the delivery sheath is introduced, such as by pushing it, through the first subject's body access in the vascular system, and/or by pulling the snare from the second subject's body access, to navigate the delivery sheath adjacent or up to the intraluminal implantation site. Then, the implantable medical device is caused to exit the delivery sheath (also referred to herein as “unsheating”).

The method 2500 may optionally include: anchoring 2504 the implantable medical device in a subject's body, such as in a lumen of a subject's body conduit like the vascular system. The first medical device component and/or the second medical device component, when provided as an anchor made of a shape-memory material (e.g., the anchors 606, 1400, 1600, 1900), may be anchored while being unsheathed from the delivery sheath, as described herein. The first medical device component and/or the second medical device component, when provided as an anchor made of a non-shape-memory material (e.g., the anchors 606, 1400, 1600, 1900), may be anchored using a balloon catheter, as also described herein.

For example, one of the first medical device component and/or the second medical device component (e.g., the anchors 606, 1400, 1600, 1900) may be anchored, while the other one of the first medical device component (e.g., the catheter 600) and/or the second medical device component may or may not be.

The method 2500 may optionally include: operating 2506 the implantable medical device in a subject's body, such as in a lumen of a subject's body conduit like the vascular system. The first medical device component and/or the second medical device component may be electrically or mechanically connected to a controller for electrically or mechanically operating the first medical device component and/or the second medical device component. One of the first medical device component and/or the second medical device component may be operated through the other one of the first medical device component and/or the second medical device component and/or the linker. The connection between the first medical device component and/or the second medical device component may be performed inside or outside the subject's body. Alternatively, the first medical device component and/or the second medical device component may be electrically or mechanically operated wirelessly.

For example, one of the first medical device component (e.g., the catheter 600) and/or the second medical device component may be electrically connected to a controller outside the subject's body to electrically operate the other one of the first medical device component and/or the second medical device component (e.g., one or more pumps) inside the subject's body.

The method 2500 may optionally include: assembling 2508 the implantable medical device in a subject's body, such as in a lumen of a subject's body conduit like the vascular system. The implantable medical device may optionally include an assemblable medical device (such as the assemblable medical device 110) configured to assemble to the first medical device component (e.g., the first medical device component 102), the second medical device component (e.g., second medical device component 104), and/or the linker of the implantable medical device. Multiple assemblable medical devices may be so assembled to the implantable medical device.

For example, the assemblable medical device, the first medical device component, and/or the second medical device component may be extracorporeally manipulated from outside the subject's body for intracorporeally assembling the assemblable medical device inside the subject's body. In particular, the assemblable medical device may be pulled relative to the first medical device component and the second medical device component, and/or the first medical device component and the second medical device component may be pushed relative to the assemblable medical device for assembling. A portion of the assemblable medical device (e.g., the elongated control structure 114 and the electrical lead 712) may be pulled from outside the subject's body, such as through the second subject's body access, for assembling the assemblable medical device (e.g., the functional unit 112) and the second medical device component (e.g., the anchors 606, 1400, 1600, 1900) together inside the subject's body. Additionally or alternatively, the second medical device component (e.g., the catheter 600) may be pushed from outside the subject's body, such as through the first subject's body access, for assembling the assemblable medical device (e.g., the functional unit 112) and the second medical device component 104X (e.g., the anchors 606, 1400, 1600, 1900) together inside the subject's body.

Still for example, the assemblable medical device, the first medical device component, the second medical device component, and/or the linker may be mechanically or electrically actuated for assembly. In particular, the elongated control element (e.g. the electrical lead 712) of the assemblable medical device may be extracorporeally or intracorporeally actuated for intracorporeally assembling the assemblable medical device (e.g., the functional unit 112) and the second medical device component (e.g., the anchors 606, 1400, 1600, 1900). The actuation may be commanded extracorporeally from outside or intracorporeally from inside the subject's body.

In an implementation, for example, the method 2500 includes: transcatheterly implanting 2502 the implantable medical device in the vascular system of the subject, such that (i) the first medical device component at least partially extends from an intraluminal implantation site up to an extracorporeal location outside the subject's body, (ii) the second medical device component is located at the intraluminal implantation site, (iii) the linker is directly or indirectly coupled to and extends between the first medical device component and the second medical device component, and (iv) the optional assemblable medical device at least partially extends from the intraluminal implantation site up to an extracorporeal location outside the subject's body. Anchoring 2504 the implantable medical device, such as the second medical device component thereof, at the intraluminal implantation site. Assembling 2508 the implantable medical device, such as the second medical device component thereof, and the assemblable medical device, such as a functional unit thereof, together at the intraluminal implantation site by manipulating the implantable medical device and/or the assemblable medical device from outside the subject's body. The implantable medical device and/or the assemblable medical device may be manipulated by pulling and/or pushing it or them for assembling. Operating 2506 the assemblable medical device, such as the functional unit thereof, at the intraluminal implantation site. The assemblable medical device may be operated from outside the subject's body by connecting it to a controller, or wirelessly.

In this last implementation, as well as any other implementations herein, implanting 2502, anchoring 2504, operating 2506, and assembling 2508 may be performed in any order, depending on the medical procedure.

As schematically represented in FIG. 26, the method 2600 of explanting an implanted medical device (e.g., the implantable medical device 100; also referred to herein as a “medical device”) from a subject's body includes: explanting 2602 the implanted medical device from the subject's body to cause a linker (e.g., the flexible linker 106) to transition from a second configuration to a first configuration, according to one or more embodiment(s).

The first and second configurations, as well as the transitioning therebetween, of the linker of the method 2600 being generally similar to the method 2500, it will appreciate that the description of the method 2500 applies to the method 2600 with the necessary change(s), appreciable to the skilled addressee, having been made, if applicable.

As described herein for the method 2500, the implanted medical device of the method 2600 may be implanted as represented in FIGS. 2-5, according to one or more embodiment(s).

Explanting 2602 may include explanting the medical device implanted in a subject vascular system according to the modalities (i) to (xvi) described for the method 2500.

Explanting 2602 may cause the linker to momentarily transition from the second configuration to the first configuration, and vice-versa. For example, this may be the case when the implantable medical device is routed in, navigated in, and/or externalized from a lumen of a subject's body conduit, causing the linker to momentarily flex, bend, articulate, pivot, hinge, or swivel for the implantable medical device to accommodate a tortuous portion of the lumen along the way.

Explanting 2602 may also cause the linker to remain transitioned from the second configuration to the first configuration, and vice-versa, for the duration of its use. For example, this may be the case when the implantable medical device is unassembled in, unanchored in, retrieved from, and/or explanted from a lumen of a subject's body conduit, causing the linker to remain so transitioned until explantation or repositioning.

Explanting 2602 may include transcatheterly explanting the implanted medical device from the vascular system of the subject's body. In particular, a medical tool, such as a snare, is introduced through the first subject's body access in the vascular system and routed therein up to an implanted medical device. The medical tool is then manipulated to capture (also referred to herein as “to snare”) an optional capturable element (e.g., the capturable element 1214) of the implanted medical device, and is pulled through the first subject's body access to retrieve or explant the implanted medical device therethrough from the subject's body. A portion of the implanted medical device (e.g., the catheter 600) that is at least partially externalized through the second subject's body access may additionally or alternatively be pushed therethrough for retrieval or explantation.

The implanted medical device may also be retrieved or explanted using an optional retrieval sheath. In particular, the retrieval sheath is railed over the medical tool up to the implanted medical device. Then, the implanted medical device is caused to enter the retrieval sheath (also referred to herein as “sheating”) by pulling the medical tool from the first subject's body access and the retrieval sheath, and/or by pushing the retrieval sheath against the implanted medical device.

The method 2600 may optionally include: unanchoring 2604 the implanted medical device from a subject's body, such as from a lumen of a subject's body conduit like the vascular system. The first medical device component and/or the second medical device component, when provided as an anchor made of a shape-memory or non-shape-memory material (e.g., the anchors 606, 1400, 1600, 1900), may be unanchored while being sheathed by the retrieval sheath, as described herein.

For example, one of the first medical device component and/or the second medical device component (e.g., the anchors 606, 1400, 1600, 1900) may be unanchored, while the other one of the first medical device component (e.g., the catheter 600) and/or the second medical device component may or may not be.

The method 2600 may optionally include: stopping 2606 the operation of the implanted medical device being electrically or mechanically operated in a subject's body, such as in a lumen of a subject's body conduit like the vascular system. The operation of the implanted medical device may be stopped via a controller and/or by disconnecting the implanted medical device from the controller. The disconnection may be performed outside or inside the subject's body. When connected to the controller, the implanted medical device may be required to be disconnected therefrom before explanting 2602, unanchoring 2604, and/or unassembling 2608 (described hereinafter).

The connection(s) of the implanted medical device of the method 2600 being generally similar to the connection(s) of the implantable medical device of the method 2500, it will appreciate that the description of the method 2500 applies to the method 2600 with the necessary change(s), appreciable to the skilled addressee, having been made, if applicable.

The method 2600 may optionally include: unassembling 2608 the implanted medical device in a subject's body, such as in a lumen of a subject's body conduit like the vascular system. The implanted medical device may optionally include an assemblable medical device (such as the assemblable medical device 110) assembled to the first medical device component (e.g., the first medical device component 102), the second medical device component (e.g., second medical device component 104), and/or the linker of the implanted medical device. Multiple assemblable medical devices may be so unassembled from the implanted medical device.

For example, the assemblable medical device, the first medical device component, and/or the second medical device component may be extracorporeally manipulated from outside the subject's body for intracorporeally unassembling the assemblable medical device inside the subject's body. In particular, the assemblable medical device may be pushed relative to the first medical device component and the second medical device component, and/or the first medical device component and the second medical device component may be pulled relative to the assemblable medical device for unassembling. A portion of the assemblable medical device (e.g., the elongated control structure 114 and the electrical lead 712) may be pushed from outside the subject's body, such as through the second subject's body access, for unassembling the assemblable medical device (e.g., the functional unit 112) and the second medical device component (e.g., the anchors 606, 1400, 1600, 1900) from each other inside the subject's body. Additionally or alternatively, the second medical device component (e.g., the catheter 600) may be pulled from outside the subject's body, such as through the first subject's body access, for unassembling the assemblable medical device (e.g., the functional unit 112) and the second medical device component 104X (e.g., the anchors 606, 1400, 1600, 1900) from each other inside the subject's body.

Additionally or alternatively, a medical tool, such as a snare, may be introduced through the first subject's body access in the vascular system and routed therein up to the assemblable medical device that is assembled to the implantable medical device. The medical tool is then manipulated to capture (also referred to herein as “to snare”) an optional capturable element (e.g., the capturable element 1214) of the assemblable medical device, and is pulled through the first subject's body access for unassembling the assemblable medical device and the second medical device component 104 from each other. The retrieval sheath may be railed over the medical tool up to the assemblable medical device. Then, the assemblable medical device is caused to enter the retrieval sheath (also referred to herein as “sheating”) by pulling the medical tool from the first subject's body access and the retrieval sheath, and/or by pushing the retrieval sheath against the assemblable medical device.

The assemblable medical device, the first medical device component, the second medical device component, and/or the linker may also be mechanically or electrically actuated for unassembling. In particular, the elongated control element (e.g. the electrical lead 712) of the assemblable medical device may be extracorporeally or intracorporeally actuated for intracorporeally unassembling the assemblable medical device (e.g., the functional unit 112) and the second medical device component (e.g., the anchors 606, 1400, 1600, 1900) from each other. The actuation may be commanded extracorporeally from outside or intracorporeally from inside the subject's body.

In an implementation, for example, the method 2600 includes: stopping 2606 the operation of the assemblable medical device, such as the functional unit thereof, being operated at the intraluminal implantation site. The assemblable medical device may be operated from outside the subject's body by connecting it to a controller, or wirelessly. Unassembling 2608 the implanted medical device, such as the second medical device component thereof, and the assemblable medical device, such as a functional unit thereof, from each other at the intraluminal implantation site by manipulating the implanted medical device and/or the assemblable medical device from outside the subject's body. The implanted medical device and/or the assemblable medical device may be manipulated by pulling and/or pushing it or them for unassembling. Unanchoring 2604 the implanted medical device, such as the second medical device component thereof, from the intraluminal implantation site. Transcatheterly explanting 2602 or transcatheterly retrieving the implanted medical device and the assemblable medical device, which is unassembled from the implanted medical device, from the vascular system of the subject.

In this last implementation, as well as any other implementations herein, stopping 2606, unassembling 2608, unanchoring 2604, and explanting 2602 may be performed in any order, depending on the medical procedure.

1. A modular transcatheterly implantable medical device, comprising:
a plurality of assemblable blood pumps, each one of the assemblable blood pumps including a pump unit and an electrical wire configured for the pump unit to be electrically operated through the electrical wire;
an anchor having a first end portion and a second end portion, the anchor being configured to intraluminally assemble to the assemblable blood pumps and to intraluminally unassemble from the assemblable blood pumps;
a catheter having a first end portion and a second end portion, the catheter being configured to be in a slidable relationship with each one of the electrical wires for intraluminally assembling the assemblable blood pumps to the anchor and for intraluminally unassembling the assemblable pump from the anchor; and
a flexible linker configured to couple the first end portion of the catheter and the second end portion of the anchor.

2. The device according to claim 1, wherein each one the electrical wires is configured to be pulled relative to the catheter for intraluminally assembling a respective pump unit to the anchor.

3. The device according to claim 2, wherein each one the electrical wires is configured to be pushed relative to the catheter for intraluminally unassembling the pump unit from the anchor.

4. The device according to claim 1, wherein each one of the pump units includes a capturable element configured to be intraluminally captured by a medical snare for intraluminally moving the pump unit.

5. The device according to claim 1, wherein the anchor has a non-expanded configuration for intraluminal transcatheter delivery of the anchor and an expanded configuration for intraluminally anchoring the anchor; the assemblable blood pumps being assemblable to the anchor only when the anchor is in the expanded configuration.

6. The device according to claim 5, wherein the anchor is self-expandable, overcomeably biased toward the expanded configuration, and has an end portion, which is opposed to the catheter, that has a tapered shape.

7. The device according to claim 1, wherein the anchor includes a plurality of assembling elements, each one of the assembling elements being configured to interact with a respective one of the electrical wires and a respective one of the pump units for intraluminally assembling a corresponding assemblable pump to the anchor and for intraluminally unassembling the corresponding assemblable pump from the anchor.

8. The device according to claim 7, wherein the assembling elements are assembling loops, each one of the assembling loops being configured to slidably receive a respective one of the electrical wires therethrough and a respective one of the pump units therethrough.

9. The device according to claim 8, wherein each one of the assembling loops is configured to engage a respective one of the pump units for intraluminally assembling the corresponding assemblable pump to the anchor and is further configured to disengage the respective pump unit for intraluminally unassembling the corresponding assemblable pump from the anchor.

10. The device according to claim 9, wherein the anchor further includes a plurality of anchoring elements, each one of the anchoring elements being configured to engage with a lumen wall.

11. The device according to claim 10, wherein each one of the anchoring elements includes a respective engagement portion configured to atraumatically engage the lumen wall, such that the anchor is moveable along the lumen wall without traumatizing the lumen wall.

12. The device according to claim 11, wherein each one of the anchoring elements defines an opening configured to slidably receive a respective one of the electrical wires therethrough.

13. The device according to claim 12, wherein the anchor further includes a helical structure.

14. The device according to claim 12, wherein the anchor further includes a stent-like structure.

15. The device according to claim 1, wherein the anchor further includes a capturable element configured to be intraluminally captured by a medical snare for intraluminally moving the anchor.

16. The device according to claim 1, wherein the second end portion of the catheter includes an atraumatic tip, the atraumatic tip being configured to be atraumatic for a lumen wall when advanced therein.

17. The device according to claim 16, wherein the atraumatic tip has a tapered shape.

18. The device according to claim 17, wherein the atraumatic tip is removable from the second end portion of the catheter for providing access to the electrical wires.

19. The device according to claim 18, wherein the atraumatic tip includes a guidewire attached thereto and projecting outwardly therefrom.

20. The device according to claim 1, wherein the flexible linker is overcomeably biased toward an initial arrangement.

21. The device according to claim 20, wherein the flexible linker is overcomeably biased toward a linear arrangement.

22. The device according to claim 20, wherein the flexible linker is overcomeably biased toward an arced arrangement.

23. The device according to claim 1, wherein the flexible linker includes one of an articulated joint, a flexible joint, a pivot point, a hinge, or a swivel.

24-30. (canceled)
